(19) United States
(12) Patent Application Publication
(10) Pub. No.: US 2025/0381260 A1
(43) Pub. Date: Dec. 18, 2025

(54) FAP2-DERIVED ANTIBODIES AND VACCINES AGAINST FUSOBACTERIUM

(71) Applicant: PROVINCIAL HEALTH SERVICES AUTHORITY, Vancouver (CA)

(72) Inventors: Robert HOLT, North Vancouver (CA); James ROUND, Vancouver (CA); Cody DESPINS, Vancouver (CA); Scott BROWN, Langley (CA)

(21) Appl. No.: 19/130,321

(22) PCT Filed: Nov. 17, 2023
(86) PCT No.: PCT/CA2023/051551

§ 371 Date: May 15, 2025

Related U.S. Application Data
(60) Provisional application No. 63/384,320, filed on Nov. 18, 2022.

(51) Int. Cl. A61K 39/114; A61K 39/00; A61K 39/40; C07K 14/195

(52) U.S. Cl. CPC A61K 39/114; A61K 39/40; C07K 14/195; A61K 2039/53; A61K 2039/6018

(57) ABSTRACT

A new composition of matter composed of engineered sequences for the expression of Fap2-derived polypeptides that provoke immunogenic responses against Fusobacterium spp. is provided. Antibodies and vaccines produced using such sequences and methods of use are also provided.

### CROSS-REFERENCE TO RELATED APPLICATIONS

This application claims priority to, and the benefit of, U.S. provisional patent application No. 63/384,320 filed 18 Nov. 2022, the entirety of which is incorporated by reference herein.

### TECHNICAL FIELD

Some embodiments relate to antigenic targets for producing antibodies and/or vaccines active against Fusobacterium spp. Some embodiments relate to antibodies or vaccines that target such antigenic targets. Some embodiments relate to vectors or constructs for expressing such antigenic targets. Some embodiments relate to therapies, including antibodies or vaccines, useful for treating cancer or other disorders or health issues including ensuring maternal health, avoiding adverse pregnancy outcomes, treating gastrointestinal disorders and other infections.

### BACKGROUND

Fusobacterium nucleatum is an invasive (Han et al. 2000, Swidsinski et al. 2011), adherent (Weiss et al. 2000) and pro-inflammatory (Peyret-Lacombe et al. 2009, Krisanaprakornkit et al. 2000) anaerobic bacterium. It is common in dental plaque (Bolstad et al. 1996, Ximenez-Fyvie et al. 2000) and there is a well established association between F. nucleatum and periodontitis (Signal et al. 2011). Anecdotally, F. nucleatum has been implicated in cerebral abscesses (Kai et al. 2008) and pericarditis (Han et al. 2003) and it is one of the Fusobacterium species implicated in Lemierre's syndrome, a rare form of thrombophlebitis (Weeks et al. 2010). Various Fusobacteria, including F. nucleatum, have been implicated in acute appendicitis, where they have been found by immunohistochemistry (IHC) as epithelial and submucosal infiltrates that correlate positively with severity of disease (Swidsinski et al. 2011). When isolated from human intestinal biopsy material, F. nucleatum has been found to be more readily culturable from patients with gastrointestinal (GI) disease than healthy controls, and the strains grown from inflamed biopsy tissue appeared to exhibit a more invasive phenotype (Strauss et al. 2008, Strauss et al. 2011).

Recent literature reviews show that F. nucleatum has been implicated in or associated with many different types of cancer and/or more adverse prognosis in various cancers including colorectal cancer (CRC), oral squamous cell carcinoma, oral/head and neck cancer, head and neck squamous cell carcinoma, esophageal cancer, esophageal squamous cell carcinoma, human papillomavirus positive oropharyngeal squamous cell carcinoma, gastric cardia adenocarcinoma, gastric cancer, Helicobacter pylori-positive gastric cancer, pancreatic cancer, stomach cancer, breast cancer, bladder cancer, cervical cancer, laryngeal squamous cell carcinoma, and lung cancer (He et al., 2022). Cancers that are positive for Fusobacterium are much more likely to relapse, the presence of a high amount of F. nucleatum has been associated with poor patient outcomes (e.g. Serna et al., 2020), and Fusobacterium may promote chemoresistance by modulating autophagy (Yu et al., 2017).

High F. nucleatum tumor burden is associated with poor patient outcomes, chemoresistance, and increased metastasis. A key virulence factor of F. nucleatum is the protein Fap2, a type Va autotransporter that mediates tumor enrichment via binding of GalGalNAc, which is upregulated by many cancer types including colorectal cancer, and which also facilitates immune inhibition via binding of TIGIT (T cell immunoreceptor with Ig and ITIM domains), which is present on T cells and NK cells.

F. nucleatum has also been implicated in a number of disorders beyond cancer, including a number of adverse pregnancy outcomes (including chorioamnionitis, preterm birth, stillbirth, neonatal sepsis, preeclampsia), gastrointestinal disorders (including inflammatory bowel disease and appendicitis), cardiovascular disease, rheumatoid arthritis, infections of the head and neck (including respiratory tract infections including Lemierre's syndrome, acute and chronic mastoiditis, chronic otitis and sinusitis, tonsillitis, peritonsillar and retropharyngeal abcesses, postanginal cervical lymphadenitis, and periodontis), as well as infections in the brain, lungs, abdomen, pelvis, bones, joints and blood, and Alzheimer's disease (Han, 2015). Studies suggest that Fap2 is involved in mediating placental localization and enrichment of F. nucleatum, which is associated with adverse pregnancy outcomes such as preterm birth (Parhi et al., 2022).

Vaccine-induced immunity against F. nucleatum may thus reduce F. nucleatum tumor burden and thereby reduce the negative clinical outcomes associated with F. nucleatum-positive cancers such as CRC. In addition, vaccination against Fusobacterium ssp. could help prevent and treat indications that are associated with Fusobacterium ssp. invasion and/or infection. Including, but not limited to: pre-term birth and miscarriages, and more broadly adverse pregnancy outcomes; other cancers, non-exhaustively including oral, head and neck, pancreatic, biliary tract, breast, and melanoma; dental disease such as periodontitis; autoimmune diseases non-exhaustively including IBD (inflammatory bowel disease), atherosclerotic disease, rheumatoid arthritis; and direct infections non-exhaustively including appendicitis, sepsis, and tissue abscesses.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

One aspect provides a target antigen, the target antigen being a Fap2 antigen having a Fap2 passenger domain from Fusobacterium spp. or an antigenic fragment thereof. The target antigen can have the sequence of the extracellular passenger domain of Fap2 or portions or fragments thereof. The target antigen can have the sequence of full length Fap2 or portions or fragments thereof, including between 8 and 3500 contiguous amino acid residues of the extracellular passenger domain of Fap 2. The target antigen can have a sequence according to any one of SEQ ID NOs: 1-5, 46-80, 97-112, 120-126 or 4560-4562 or fragments thereof. The target antigen can be a B-cell epitope having a sequence according to any one of SEQ ID NOs: 127-294 or T cell epitope having a sequence according to any one of SEQ ID NOs: 317-4577. The target antigen can have an N-terminal secretion signal having a sequence according to any one of SEQ ID NOs: 295-316. The target antigen can have a transmembrane domain or C-terminal multimerization domain.

One aspect provides isolated nucleic acid molecules encoding the target antigens described above and polypeptides having any of the sequences set forth above. The nucleic acid molecules can be DNA or mRNA. The nucleic acid molecules can have a sequence according to any one of SEQ ID NOs: 6-10, 11-45, 81-96, or 113-119.

One aspect provides a vaccine including the target antigens described above or the nucleic acid molecules described above. The vaccine can have a nucleotide construct encoding the target antigens as described above, the nucleotide construct can be DNA or mRNA. The vaccine can have an mRNA construct where the mRNA is formulated in a lipid nanoparticle. The vaccine can be a viral vector vaccine or a DNA plasmid vaccine.

One aspect provides an antibody targeting the target antigens as described above. One aspect provides an antibody produced using the target antigens as described above.

One aspect provides use of the target antigens, nucleic acid molecules, vaccines or antibodies described above to induce an immunological response against Fusobacterium spp. in a subject.

The target antigens described herein can be used in the prevention and treatment of cancers involving Fusobacterium spp. The target antigens described herein can be used to prevent and treat adverse pregnancy outcomes, dental disease and autoimmune disease involving Fusobacterium spp. The target antigens described herein can be used to prevent and treat conditions caused by or related to infection by Fusobacterium spp.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 shows the predicted structure of Fap2 and its associated domains.

FIG. 2 shows the expression of Fap2 antigens in eukaryotic cell culture using intracellular staining and flow cytometry.

FIG. 3 shows detection of Fap2-specific antibodies in plasma pools of Fap2 mRNA-LNP immunized mice.

FIG. 4 shows detection of Fap2-specific antibodies in plasma pools of Fap2 mRNA-LNP immunized mice for additional Fap2 antigen constructs.

FIG. 5 shows flow cytometry detection of surface FLAG expression in FLAG-tagged transmembrane displayed Fap2 truncation pDNA transfectants.

FIG. 6 shows flow cytometry detection of StrepTagII expression in Strep-tagged transmembrane displayed Fap2 truncation pDNA transfectants.

FIG. 7 shows reactivity of secreted and transmembrane displayed Fap2 truncation vaccine serum pools to Fap2-T3.

FIG. 8 shows the reactivity of B-cell hybridoma media samples to various Fap2 antigen sources.

FIG. 9 shows the per amino acid B-cell epitope probability scores as overlaid on the predicted structure of Fap2.

FIG. 10 shows plots of the per amino acid B-cell epitope probability scores for Fap2 in nine F. nucleatum subspecies.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

As used herein, the term “cancer” or “neoplasm” refers to any unwanted growth of cells serving no physiological function. In general, a cell of a neoplasm has been released from its normal cell division control, i.e., a cell whose growth is not regulated by the ordinary biochemical and physical influences in the cellular environment. In most cases, a neoplastic cell proliferates to form a clone of cells that are either benign or malignant. Examples of cancers or neoplasms include, without limitation, transformed and immortalized cells, tumours, and carcinomas such as breast cell carcinomas and prostate carcinomas. The term cancer includes cell growths that are technically benign but which carry the risk of becoming malignant i.e. a “malignancy.” The term “malignancy” refers to an abnormal growth of any cell type or tissue. The term malignancy includes cell growths that are technically benign, but which carry the risk of becoming malignant. This term also includes any cancer, carcinoma, neoplasm, neoplasia, or tumor.

As used herein, the terms “gastrointestinal” or “GI” cancer or carcinoma refers to a malignancy or neoplasm of the gastrointestinal tract. GI cancers can include cancers of the upper GI tract such as, esophagus (e.g., squamous cell carcinoma, adenocarcinoma), or stomach (e.g., gastric carcinoma, signet ring cell carcinoma, gastric lymphoma) or of the lower GI tract such as, small intestine (e.g., duodenal cancer/adenocarcinoma), colon/rectum (e.g., colorectal polyps/Peutz-Jeghers syndrome, juvenile polyposis syndrome, familial adenomatous polyposis/Gardner's syndrome, Cronkhite-Canada syndrome, familial adenomatous polyposis, hereditary nonpolyposis colorectal cancer, etc.), anus (e.g., squamous cell carcinoma).

As used herein, the term “Fusobacterium” refers to a genus of gram-negative, anaerobic, rod-shaped bacteria found as normal flora in the mouth and large bowel and often in necrotic tissue (Miller-Keane Encyclopedia and Dictionary of Medicine, Nursing, and Allied Health, Seventh Edition. 2003 by Saunders, an imprint of Elsevier, Inc.). Some Fusobacterium species are pathogenic to humans (Mosby's Medical Dictionary, 8th edition. 2009, Elsevier). Fusobacterium species include F. gonidiaformans and F. mortiferum (occurring in respiratory, urogenital, and gastrointestinal infections); F. necrophorum (occurring in disseminated infections involving necrotic lesions, abscesses, and bacteremia), F. naviforme, F. russii, and F. varium (occurring in abscesses and other infections), F. fusiforme (found in cavities of humans and other animals, and sometimes associated with Vincent's angina), F. polymorphum, F. equinum, F. nodosus, F. nucleatum, etc. (Miller-Keane Encyclopedia and Dictionary of Medicine, Nursing, and Allied Health, Seventh Edition. © 2003 by Saunders, an imprint of Elsevier, Inc.; Mosby's Medical Dictionary, 8th edition. © 2009, Elsevier). In some embodiments, a Fusobacterium species includes a Fusobacterium sp. strain 3_1_36A2, Fusobacterium sp. strain 3_1_27, Fusobacterium sp. strain 7_1, Fusobacterium sp. strain 4_1_13, Fusobacterium sp. strain D11, Fusobacterium sp. strain 3_1_33, F. gonidiaformans ATCC 25563, Fusobacterium sp. strain 1_1_41FAA, etc.

As used herein, the term “Fusobacterium nucleatum” or “F. nucleatum” is meant as an invasive, adherent and pro-inflammatory anaerobic bacterium. In some embodiments, a F. nucleatum includes a F. nucleatum subsp. nucleatum ATCC 25586, F. nucleatum subsp. polymorphum ATCC 10953, Fusobacterium sp. strain 3_1_36A2, F. nucleatum CC53, Fusobacterium sp. strain 3_1_27, F. nucleatum subsp. vincentii ATCC 49256, F. nucleatum 7/1, Fusobacterium sp. strain 4_1_13, Fusobacterium sp. strain D11, F. nucleatum subsp. nucleatum ATCC 23726, Fusobacterium sp. strain 3_1_33, Fusobacterium sp. strain 1_1_41FAA, etc.

In some embodiments, the F. nucleatum subsp. nucleatum ATCC 25586 has a nucleic acid sequence substantially identical to one or more of the sequences referenced in GenBank Accession No. AE009951 or to NC_003454.1 or a fragment or variant thereof. In some embodiments, the F. nucleatum subsp. polymorphum ATCC 10953 has a nucleic acid sequence substantially identical to one or more of the sequences referenced in GenBank Accession No. NZ_CM000440, or a fragment or variant thereof. In some embodiments, the Fusobacterium sp. strain 3_1_36A2 has a nucleic acid sequence substantially identical to one or more of the sequences referenced in GenBank Accession Nos. ACPU01000001 to ACPU01000051, or GG698790-GG698801, or a fragment thereof. In some embodiments, the F. nucleatum 7/1 has a nucleic acid sequence substantially identical to the sequence referenced in GenBank Accession No. CP007062.1, or a fragment thereof. In some embodiments, the F. nucleatum ATCC 23726 has a nucleic acid sequence substantially identical to the sequence referenced in GenBank Accession No. NZ_CP028109.1, or a fragment thereof.

Given that Fap2 is a suspected virulence factor for Fusobacterium spp., the inventors hypothesized that generating anti-Fap2 immunity with a vaccine may induce Fap2-specific neutralizing antibodies, thereby targeting an immune response against Fusobacterium spp. and preventing tumor enrichment and immune inhibition, and evoke a CD8⁺ T cell response, targeting Fusobacterium spp. invaded host cells.

In some embodiments, the inventors have created new compositions of matter composed of engineered sequences, or constructs, for the expression of Fap2-derived polypeptides, or antigens, that provoke immunogenic responses against Fusobacterium spp. and are thus amenable to the design of a vaccine. In some embodiments, these constructs are cloned into vectors that allow for in vitro transcription of mRNA and the plasmid-borne production of the Fap2-derived antigens in eukaryotic cells. In some embodiments, these constructs include high homology regions that provide immunogenicity against different Fusobacterium species and subspecies. In some embodiments, the Fusobacterium spp. is F. nucleatum. In some embodiments, the F. nucleatum is F. nucleatum 7/1, F. nucleatum ATCC23726, F. nucleatum ChDC-F317, F. nucleatum Fn3-1-27, F. nucleatum Fn3-1-36A2, F. nucleatum Fn4-8, F. nucleatum Fn71, F. nucleatum KCOM-1322, F. nucleatum KCOM-2931, and/or F. nucleatum MGYG-HGUT-01347.

In one embodiment, a target antigen derived from Fap2 is provided. In some embodiments, the target antigen is a region of the extracellular passenger domain of Fap2. In some embodiments, the target antigen contains between 8 and 3500 contiguous amino acid residues of the extracellular passenger domain of Fap2 , including e.g. 9, 10, 11, 12, 13, 14, 15, 16, 17, 18, 19, 20, 21, 22, 23, 24, 25, 26, 27, 28, 29, 30, 40, 50, 75, 100, 200, 300, 400, 500, 600, 700, 800, 900, 1000, 1200, 1400, 1600, 1800, 2000, 2250, 2500, 2750, 3000 or 3250 contiguous amino acid residues of the extracellular passenger domain of Fap2. In some embodiments, any portion of the extracellular passenger domain of Fap2 that is at least 8 contiguous amino acids in length represents a potential epitope for cytolytic CD8+ T cells.

In some embodiments, the target antigen has an amino acid sequence having along its length between 90% and 100% sequence identity to the corresponding portion of the reference sequence of Fap2 from F. nucleatum 7/1 shown in SEQ ID NO:1, including any value therebetween e.g. 91%, 92%, 93%, 94%, 95%, 96%, 97%, 97.5%, 98%, 98.5%, 99%, 99.5% or 99.9%. While throughout this specification amino acid residues are described with reference to the corresponding position of the reference protein sequence of Fap2 from F. nucleatum 7/1, those skilled in the art will appreciate that Fap2 sequences may differ slightly between Fusobacterium spp. so that the specific positions of the amino acid residues in a different Fusobacterium species should be determined with reference to the amino acid residues that correspond to the positions identified herein for the Fap2 reference protein sequence from F. nucleatum 7/1.

In some embodiments, the target antigen has the amino acid sequence of one of constructs shown in Table 1:

TABLE 1
Sequence of tested Fap2 target antigens.

| | | | Corresponding | |
|---|---|---|---|---|
| | | | Amino Acid | |
| | | | Residues of | |
| Name of | Amino Acid | DNA | Fap2 from F. | |
| Construct, | Positions of | Sequence | nucleatum 7/1 | |
| Features of | Corresponding | Amino | (3799 aa in | Corresponding |
| Construct | Features | Acid Seq. | length) | Sequence |
| FL (full | 1-21: IGk signal | SEQ ID | 43-3495 | Amino acid |
| length) Fap2, | 22-3474: FL-Fap2 | NO: 6 | | residues 22-3474 |
| with Ig kappa | domain | SEQ ID | | of SEQ ID NO: 1 |
| signal | 3475-3484: | NO: 1 | | |
| peptide and | Strep-tag | | | |
| strep-tag | | | | |
| T1, with Ig | 1-21: IGk signal | SEQ ID | 43-371 | Amino acid |
| kappa signal | 22-350: T1-Fap2 | NO: 7 | | residues 22-350 |
| peptide and | domain | SEQ ID | | of SEQ ID NO: 2 |
| strep-tag | 354-363: | NO: 2 | | |
| | Strep-tag | | | |
| T2, with Ig | 1-21: IGk signal | SEQ ID | 43-1080 | Amino acid |
| kappa signal | 22-1059: T2-Fap2 | NO: 8 | | residues 22-1059 |
| peptide and | domain | SEQ ID | | of SEQ ID NO: 3 |
| strep-tag | 1063-1072: | NO: 3 | | |
| | Strep-tag | | | |
| T3, with Ig | 1-21: IGk signal | SEQ ID | 43-1627 | Amino acid |
| kappa signal | 22-1606: T3-Fap2 | NO: 9 | | residues 22-1606 |
| peptide and | domain | SEQ ID | | of SEQ ID NO: 4 |
| strep-tag | 1610-1619: | NO: 4 | | |
| | Strep-tag | | | |
| T4, with Ig | 1-21: IGk signal | SEQ ID | 43-2274 | Amino acid |
| kappa signal | 22-2252: T4-Fap2 | NO: 10 | | residues 22-2252 |
| peptide and | domain | SEQ ID | | of SEQ ID NO: 5 |
| strep-tag | 2257-2266: | NO: 5 | | |
| | Strep-tag | | | |

In some embodiments, the target antigen is one of the FL, T1, T2, T3 or T4 constructs listed above in Table 1, which correspond respectively to amino acid residues 22-3474, 22-350, 22-1059, 22-1606 or 22-2252 of SEQ ID NO:1. In some embodiments, the target antigen contains a portion of one of the constructs listed above with a portion corresponding to one of the shorter constructs listed above removed; for example, FLΔT4, FLΔT3, FLΔT2 or FLΔT1, T4ΔT3, T4ΔT2, T4ΔT1, T3ΔT2, T3ΔT1 or T2ΔT1, wherein the first referenced construct reflects the starting construct and the second referenced construct following the A represents the portion of the starting construct that is deleted to arrive at the recited fragment (which constructs correspond respectively to amino acid residues 2253-3474, 1607-3474, 1060-3474, 351-3474, 1607-2252, 1060-2252, 351-2252, 1060-1606, 351-1606 or 351-1059 of SEQ ID NO:1). In some embodiments, the target antigens are T2ΔT1 or T3ΔT1, which correspond to amino acid residues 372-1080 and 372-1627, respectively, of the reference protein Fap2 from F. nucleatum 7/1 (which constructs correspond respectively to amino acid residues 351-1059 and 351-1606 of SEQ ID NO:1). In some embodiments, the target antigen contains between 8 and 546 contiguous amino acid residues of the extracellular passenger domain of Fap2 extending between positions 1081 and 1627 of the Fap2 protein sequence from F. nucleatum 7/1 (which corresponds to amino acid residues 1060-1606 of SEQ ID NO: 1) including any value or subrange therebetween e.g. 9, 10, 11, 12, 13, 14, 15, 16, 17, 18, 19, 20, 21, 22, 23, 24, 25, 26, 27, 28, 29, 30, 40, 50, 75, 100, 200, 300, 400, or 500 contiguous amino acid residues. In some embodiments, the target antigen contains between 8 and 1256 contiguous amino acid residues of the extracellular passenger domain of Fap2 extending between positions 371 and 1627 of the reference Fap2 protein sequence from F. nucleatum 7/1 (which corresponds to amino acid residues 350 to 1606 of SEQ ID NO: 1) including any value or subrange therebetween, e.g. 9, 10, 11, 12, 13, 14, 15, 16, 17, 18, 19, 20, 21, 22, 23, 24, 25, 26, 27, 28, 29, 30, 40, 50, 75, 100, 200, 300, 400, 500. 600, 700, 800, 900, 1000, 1100, 1200, 1300, 1400, 1500 or 1600 contiguous amino acid residues.

In some embodiments, the target antigen has the amino acid sequence of any one of SEQ ID NOs: 1-5. In some embodiments, the target antigen has the amino acid sequence of any one of SEQ ID NOs: 46-80, 97-112, 120-126, or 4560-4562.

In some embodiments, the target antigen is a B-cell epitope. In some such embodiments, the target antigen has an amino acid sequence corresponding to any one of SEQ ID NOs: 127-294 shown in Table 3.

In some embodiments, the target antigen is a T-cell epitope, including a CD8+ T-cell epitope. In some such embodiments, the target antigen has an amino acid sequence corresponding to any one of SEQ ID NOs: 317-4557.

In some embodiments, the target antigens are engineered to enhance the ability of the target antigen to generate an antigenic response in a mammal, including in a human. For example, in some embodiments, the target antigens are coupled to a suitable transmembrane domain to facilitate presentation of the target antigen to stimulate an immune response in a mammal, including in a human.

In some embodiments, the target antigens are coupled to an N-terminal secretion signal to facilitate secretion of the target antigens by mammalian cells, including by human cells. In some embodiments, the signal peptide is one of: chymotrypsinogen, trypsinogen-2, interleukin-2, serum albumin preproprotein, immunoglobulin heavy chain, immunoglobulin light chain, azurocidin preproprotein, cystatin-S precursor, Ig kappa light chain precursor (mutant A2), oncostatin-M, glycoprotein G, Ig kappa chain V-III, Ig heavy chain V, SPARC, secrecon, Ig kappa chain V-I, myeloid cell surface antigen CD33, tissue-type plasminogen activator, gaussia luciferase, influenza haemagglutinin, insulin, silkworm fibroin light chain. In some embodiments, the N-terminal secretion signal is an Ig kappa signal peptide. In some embodiments, the signal peptide has one of the sequences set forth in Table 4.

TABLE 4
Signal peptide sequences.

| | Species of | | |
|---|---|---|---|
| Signal Peptide | Origin | Sequence | SEQ ID NO: |
| Chymotrypsinogen | Homo sapiens | MAFLWLLSCWALLGTTFG | SEQ ID NO: 295 |
| Trypsinogen-2 | Homo sapiens | MNLLLILTFVAAAVA | SEQ ID NO: 296 |
| Interleukin-2 | Homo sapiens | MYRMQLLSCIALSLALVTNS | SEQ ID NO: 297 |
| Serum albumin | Homo sapiens | MKWVTFISLLFLFSSAYS | SEQ ID NO: 298 |
| preproprotein | | | |
| Immunoglobulin | Homo sapiens | MDWTWRVFCLLAVTPGAHP | SEQ ID NO: 299 |
| heavy chain | | | |
| Immunoglobulin | Homo sapiens | MAWSPLFLTLITHCAGSWA | SEQ ID NO: 300 |
| light chain | | | |
| Azurocidin | Homo sapiens | MTRLTVLALLAGLLASSRA | SEQ ID NO: 301 |
| preproprotein | | | |
| Cystatin-S | Homo sapiens | MARPLCTLLLLMATLAGALA | SEQ ID NO: 302 |
| precursor | | | |
| Ig kappa light | Mus musculus | MDMRAPAGIFGFLLVLFPGYRS | SEQ ID NO: 303 |
| chain precursor | | | |
| (mutant A2 | | | |
| Oncostatin-M | Homo sapiens | MGVLLTQRTLLSLVLALLFPSMASM | SEQ ID NO: 304 |
| Glycoprotein G | Vesicular | MKCLLYLAFLFIGVNC | SEQ ID NO: 305 |
| | stomatitis | | |
| | Indiana virus | | |
| Ig kappa chain | Mus musculus | METDTLLLWVLLLWVPGSTG | SEQ ID NO: 306 |
| V-III | | | |
| Ig heavy chain V | Mus musculus | MGWSCIILFLVATATGVHS | SEQ ID NO: 307 |
| SPARC | Homo sapiens | MRAWIFFLLCLAGRALA | SEQ ID NO: 308 |
| Secrecon | Synthetic | MWWRLWWLLLLLLLLWPMVWA | SEQ ID NO: 309 |
| Ig kappa chain V-I | Homo sapiens | MDMRVPAQLLGLLLLWLRGARC | SEQ ID NO: 310 |
| Myeloid cell | Homo sapiens | MPLLLLLPLLWAGALA | SEQ ID NO: 311 |
| surface antigen | | | |
| CD33 | | | |
| Tissue-type | Homo sapiens | MDAMKRGLCCVLLLCGAVFVSPS | SEQ ID NO: 312 |
| plasminogen | | | |
| activator | | | |
| Gaussia luciferase | Gaussia | MGVKVLFALICIAVAEA | SEQ ID NO: 313 |
| | princeps | | |
| Influenza | Influenza A | MKTIIALSYIFCLVLG | SEQ ID NO: 314 |
| Haemagglutinin | virus | | |
| Insulin | Homo sapiens | MALWMRLLPLLALLALWGPDPAAA | SEQ ID NO: 315 |
| Silkworm Fibroin | Bombyx mori | MKPIFLVLLVVTSAYA | SEQ ID NO: 316 |
| light chain | | | |

In some embodiments, the target antigens are engineered to enhance the valency of the target antigen. For example, in some embodiments, the target antigens are coupled to a C-terminal transmembrane or multimerization domain to increase antigen valency. Non-limiting examples of potential transmembrane or multimerization domains that can be used to increase antigen valency include transmembrane anchors derived from T3(10), O3(33), Nsp10, Lumazine Synthase, M1 VLP, I3 (01), I52 (32), I53 (50), I32 (28), HbsAg VLP, PDGFR or B7-1, or self-assembling domains that can be used for the creation of protein nanoparticles, for example Foldon, Ferritin, E2p, mi3, AP205, or IMX313. In some embodiments, alternative transmembrane domains such as the transmembrane domains from CD28, CD8, CD86, FasL, IgM or the like are used.

In some embodiments, nucleic acid constructs encoding the amino acid sequence of any of the foregoing target antigens, including the foregoing engineered target antigens, are provided. In some embodiments, the nucleic acid constructs are DNA constructs, for example suitable vectors for expressing the target antigens, e.g. in a mammalian cell, including in a human cell. In some embodiments, the nucleic acid constructs are mRNA constructs capable of expressing the target antigens, e.g. in a mammalian cell, including in a human cell.

In some embodiments, the nucleic acid constructs have the nucleotide sequence of any one of SEQ ID NOs: 6-10, 11-45, 81-96, or 113-119.

In some embodiments, the target antigen or a nucleic acid construct encoding the target antigen is used to stimulate an immune response in a mammal, including in a human. In some embodiments, the target antigen or nucleic acid construct encoding the target antigen is administered to a subject as a vaccine, to stimulate an immune response against Fusobacterium spp. in the subject. In various embodiments, any suitable type of vaccine can be used to deliver the target antigen to a subject to stimulate an immune response against Fusobacterium spp., for example an mRNA vaccine; a viral vector vaccine; a DNA plasmid vaccine, or any other suitable type of vaccine currently known or developed in future.

In some embodiments, the target antigen or a nucleic acid construct encoding the target antigen is used to produce an antibody, for example a monoclonal antibody. The antibody is then administered to a subject to stimulate an immune response against Fusobacterium spp. in the subject, for example to treat cancer or other disorders.

In some embodiments, target antigens, vaccines and/or antibodies as described herein are administered to a subject to induce an immunological response against Fusobacterium spp. In some embodiments, target antigens, vaccines and/or antibodies as described herein are administered to a subject to prevent or treat a cancer. Specifically, since Fusobacterium spp. is implicated in chemoresistance, cancer recurrence, adverse outcomes, poor patient prognosis and the like, achieving a reduction or elimination of the Fusobacterium spp. can help to treat a cancer, including a chemoresistant cancer, can help to prevent recurrence of the cancer, can improve patient outcomes, can improve patient prognosis, and the like. In some embodiments, a reduction or elimination of Fusobacterium spp. is achieved by administering to a subject a target antigen, a vaccine, or an antibody as described in this specification. In some embodiments, the administration of such a target antigen, vaccine or antibody can prevent or mitigate chemoresistance of Fusobacterium spp. positive cancers, can prevent re-colonization of a cancer with Fusobacterium spp., can extend a period of cancer remission in a patient that has received treatment for the cancer, and/or can help to prevent metastatic spread of a localized cancer which may be facilitated by Fusobacterium spp.

In some embodiments, a target antigen, vaccine and/or antibody as described herein is administered to a subject in conjunction with a conventional cancer therapy (e.g. surgery, chemotherapy and/or radiation therapy). Because the target antigen, vaccine and/or antibody acts to reduce or eliminate Fusobacterium spp. and can therefore limit the negative effects that the presence of this bacteria can have in cancer patients, such treatment can improve outcomes for the cancer patient. In some such embodiments, the target antigen, vaccine and/or antibody can be administered as an adjuvant therapy to the cancer treatment (i.e. as an additional treatment given after the primary cancer treatment has been provided). In other such embodiments, the target antigen, vaccine and/or antibody can be administered as a neoadjuvant therapy to the cancer treatment (i.e. as an additional treatment administered prior to the primary cancer treatment is provided).

In some embodiments, the cancer is colorectal cancer (CRC), oral squamous cell carcinoma, oral/head or neck cancer, head and neck squamous cell carcinoma, esophageal cancer, esophageal squamous cell carcinoma, human papillomavirus positive oropharyngeal squamous cell carcinoma, gastric cardia adenocarcinoma, gastric cancer, Helicobacter pylori-positive gastric cancer, pancreatic cancer, stomach cancer, breast cancer, bladder cancer, cervical cancer, laryngeal squamous cell carcinoma, lung cancer, biliary tract cancer, or melanoma. In some embodiments, the cancer is gastrointestinal cancer. In some embodiments, the gastrointestinal cancer is colorectal cancer.

In some embodiments, given the role that Fusobacterium spp. can play in driving adverse pregnancy outcomes, achieving a reduction or elimination of the Fusobacterium spp. can ameliorate or avoid such an adverse pregnancy outcome. In some embodiments, the reduction or elimination of Fusobacterium spp. is achieved by the administration to a subject of a target antigen, a vaccine or an antibody as disclosed in this specification. In some embodiments, the adverse pregnancy outcomes that are avoided by such administration can include pre-term birth, miscarriages, chorioamnionitis, neonatal sepsis, or preeclampsia.

In some embodiments, given the role that Fusobacterium spp. can play in dental diseases including periodontitis, autoimmune diseases including irritable bowel syndrome, atherosclerotic disease, rheumatoid arthritis, and various infections including appendicitis, sepsis or tissue abscesses, a method of treating such disease or infection is provided in which a target antigen, a vaccine or an antibody as disclosed in this specification is administered to a subject to achieve a reduction or elimination of Fusobacterium spp. in the subject.

In some embodiments, traditional antibacterial treatments such as antibiotics can be used in combination with the target antigens, vaccines and/or antibodies as disclosed in this specification to provide a combination therapy for reducing or eliminating Fusobacterium spp. in a subject. For example, a suitable antibiotic such as metronidazole is administered to the subject to reduce or eliminate an infection or colonization of Fusobacterium spp. A target antigen and/or vaccine as described in this specification is then administered to the subject to prevent re-infection or re-colonization of the Fusobacterium spp. in the subject.

In some embodiments, the administration of a target antigen, vaccine and/or antibody as disclosed in this specification to a subject induces production of Fap2-specific neutralizing antibodies by the subject and/or evokes a CD8+ T cell response that targets host cells that have been invaded by Fusobacterium spp.

In some embodiments, the administration of a target antigen, vaccine and/or antibody as disclosed in this specification to a subject prevents immunosuppression in the subject that can be caused by Fusobacterium spp. via a Fap2 blockade of TIGIT (T cell immunoreceptors with Ig and ITIM domains) in the subject.

In some embodiments, the administration of a target antigen, vaccine and/or antibody as disclosed in this specification to a subject disrupts an interaction between Fap2 of Fusobacterium spp. and a GalGal-Nac molecule within the subject.

In some embodiments, the Fusobacterium spp. is F. nucleatum. In some embodiments, the Fusobacterium spp. is F. nucleatum 7/1, F. nucleatum ATCC23726, F. nucleatum ChDC-F317, F. nucleatum Fn3-1-27, F. nucleatum Fn3-1-36A2, F. nucleatum Fn4-8, F. nucleatum Fn71, F. nucleatum KCOM-1322, F. nucleatum KCOM-2931, or F. nucleatum MGYG-HGUT-01347.

In some embodiments, the subject is a mammalian subject. In some embodiments, the subject is a human subject.

### EXAMPLES

Certain embodiments are further described with reference to the following examples, which are intended to be illustrative and not limiting in nature.

### Example 1.0

### Structure-Based Approach to Design Fap2-Derived Antigens

Type Va autotransporters are large and extremely complicated polypeptides. Therefore, to create Fap2-derived antigens amenable to the design of a vaccine, a structure-based approach was employed. Alphafold2 was used to generate an in silico prediction of the Fap2 structure from Fusobacterium ssp.

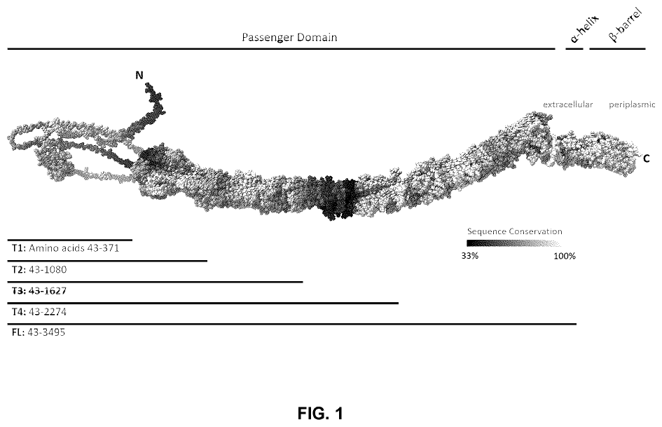

FIG. 1 shows the predicted structure of Fap2 and its associated domains based on the structure-based approach using Alphafold2. The full Fap2 protein sequence from F. nucleatum 7/1 is 3799 amino acids long. To make the structure prediction computationally tractable, the 41 amino acid N-terminal signal sequence was first removed, and the resulting 3758 amino acid protein was broken into four 2000 amino acid fragments overlapping by 800 amino acids (except for the last fragment which overlapped by 1843 amino acids). Each fragment was analyzed using AlphaFold2 and the highest confidence model for each fragment was selected. The overlapping sections were aligned, trimmed, and merged using ChimeraX to generate the full-length structure, which is comprised of the extracellular passenger domain, an α-helical linker, and a transmembrane β-barrel (these domains are marked at the top of the FIG. 1 by horizontal black lines). The predicted structure of the amino acid strand located at the N-terminal of the passenger domain had low confidence, and thus the structure of the strand was predicted independently and manually added to the remainder of the assembled protein structure.

### Example 2.0

### Antigen Selection and Preparation of Constructs

Based on the predicted structure of Fap2, the passenger domain was selected as the vaccine antigen. Sequence conservation was measured across nine F. nucleatum subsp., measuring the number of positions in a 51 amino acid sliding window where all nine subsp. had the same amino acid sequences. Sequence conservation of Fap2 between the nine F. nucleatum subsp. are shown in the structure of FIG. 1 using shading-white for an exact match for all subsp. and black for 33% of amino acids matching in all subsp. Overall, 78.2% of sites are identical across a multiple sequence alignment of Fap2 from F. nucleatum 7/1, ATCC23726, ChDC-F317, Fn3-1-27, Fn3-1-36A2, Fn4-8, KCOM-1322, KCOM-2931 and MGYG-HGUT-01347.

Five truncations of the Fap2 passenger domain were designed and constructed which contain regions of varying strain-specificity. These truncations are demarcated by the black horizontal lines at the bottom of FIG. 1 as T1, T2, T3, T4, and “Full Length” [FL]. Designs were codon-optimized with regards to both expression in human cells and the best-practices of mRNA design. In addition, an N-terminal secretion signal (Ig kappa signal peptide) and a C-terminal strep-tag were added to the designs, facilitating antigen secretion and antigen detection/purification, respectively. The resulting constructs were synthesized as DNA fragments and assembled using a BsaI-based Golden Gate Assembly reaction. Resulting sequences were then assembled using a paqCl-based Golden Gate Assembly reaction into a plasmid containing: a CMV promoter, for plasmid-borne expression in eukaryotic cells; a T7 promoter, for the in vitro transcription of mRNA; as well as, an α-globin 5′-UTR, a tandem β-globin 3′UTR, and a bisected poly (A) tail.

### Example 3.0

### Expression of Antigens in Eukaryotic Cells

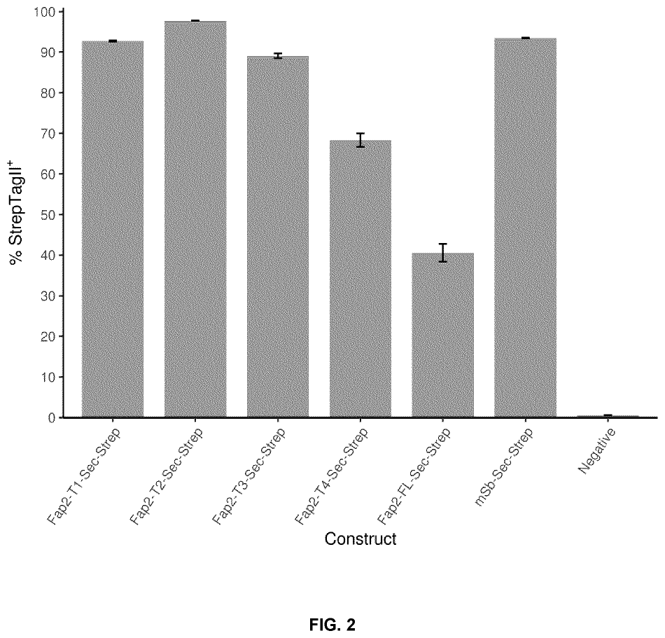

As shown in FIG. 2, flow cytometry detection of StrepTagII expression in secreted Fap2 truncation (T1, T2, T3, T4 and FL from F. nucleatum 7/1) pDNA transfectants was carried out. HEK293T/17 cells were plated at 2.5ml/well (540,000 cells/ml) in 6-well plates ˜18 hours prior to transfection. Cells were transfected with ˜2.5 ug of plasmid DNA using TransIT-LT1. Cells were harvested for analysis at ˜48 hours following transfection. Cells were stained with Fixable Viability Dye eFluor780 for 30 mins, fixed with 4% formaldehyde for 15 mins, permeabilized with cell permeablization buffer for 10 mins, and then stained with FITC-conjugated mouse anti-StrepTagII antibody for 30 mins. Cells were analyzed on a BD Fortessa flow cytometer. Resulting events were gated to isolate singlet live cells, and StrepTagII-positivity was determined relative to an unstained control. All transfections were performed in duplicate. Bars represent the mean StrepTagII-positivity, and error bars represent the standard error of the mean.

As shown in FIG. 2, all designs expressed their antigen. Without being bound, expression level of antigen appeared to be size-dependent for the tested constructs.

### Example 4.0-Testing immunogenicity of mRNA-LNP delivered Fap2 antigens

To test the immunogenicity of mRNA-LNP delivered Fap2 antigens, a boost-prime strategy was employed in mice. An in vivo experiment was designed to test mRNA-LNP delivery of Fap2-derived antigens for immunogenicity. Female HLA-A2 (C57BL/6-MCPH1-Tg (HLA-A2.1) 1Enge/J strain) humanized mice (n=4-5 per group) were vaccinated with 1 μg of Fap2 mRNA-LNP complexes or RFP mRNA-LNP complexes (negative control) using a prime-boost regimen. Specifically, mice were injected with 1 ug prime vaccine doses on day 0. Subsequently, 21 days after prime inoculation, mice were injected with 1 ug boost vaccine doses. Finally, 14 days after boost inoculation, mice were euthanized and samples were collected for evaluation of immunogenicity against F. nucleatum. Vaccinations were given via IM injection. Blood samples were collected and processed to isolate plasma or serum, as noted.

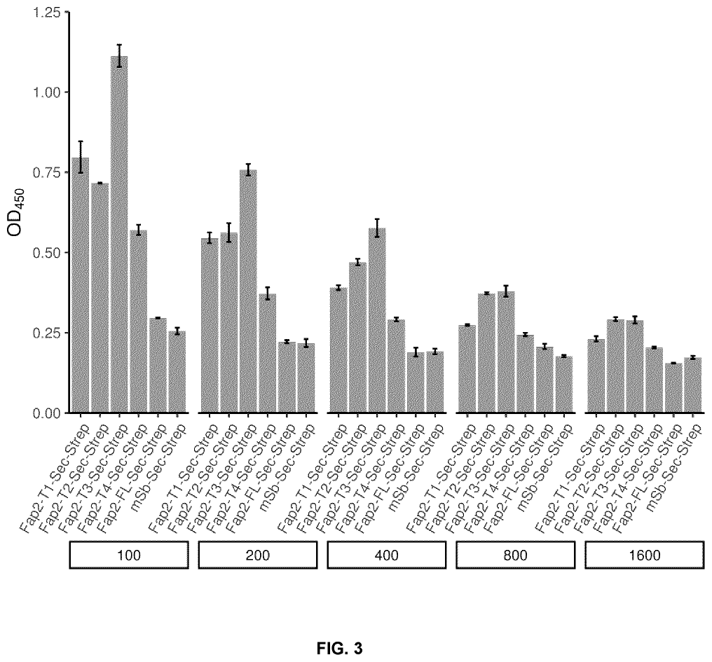

In accordance with the strategy summarized above and as shown in FIG. 3, plasma was harvested from the Fap2 mRNA-LNP immunized mice at week 4 (i.e. 14 days after boost inoculation) and examined for Fap2-specific antibodies. The reactivity of the secreted Fap2 truncation vaccine plasma pools to Fap2-FL was evaluated.

To examine for Fap2-specific antibodies, HEK293T/17 cells were transfected with Fap2-FL-Sec-Strep plasmid DNA using TransIT-LT1. At ˜48 hours following transfection, transfectant media was collected. Transfectant media was then used to coat pre-blocked streptavidin-coated 96-well plates (95 ul of media/well) after washing the plates 3× with ELISA wash buffer. Plates were incubated for 90 mins at 4 C to allow for antigen binding. Following coating, plates were washed 3× with ELISA wash buffer and 100 ul of diluted plasma samples were added to wells in duplicate. Plates were incubated at 4 C overnight. Following overnight incubation, the plates were washed 5× with ELISA wash buffer, and 100 ul of HRP-conjugated goat anti-mouse IgG antibody (1/2000 dilution) was added per well and allowed to incubate at RT for 2.5 hours. Plates were washed 5× with ELISA wash buffer, and 100 ul of TMB substrate solution was added per well. Reactions were allowed to develop for ˜20 mins, then 100 ul of ELISA stop solution was added and OD450 was measured. Each plasma sample was assayed in duplicate. Facets in FIG. 3 represent dilutions of plasma (100-fold, 200-fold, 400-fold, 800-fold, or 1600-fold). Bars represent the mean OD450, and error bars represent the standard error of the mean.

As shown in FIG. 3, relative to controls, immunization with Fap2-derived antigens resulted in antibodies that could specifically detect recombinant Fap2. The negative control was mSb (mStrawberry red fluorescent protein). The results of this example demonstrate that the tested target Fap2 antigens (T1, T2, T3, T4 and FL from F. nucleatum 7/1) are able to stimulate an immune response when delivered in vivo to a mammal.

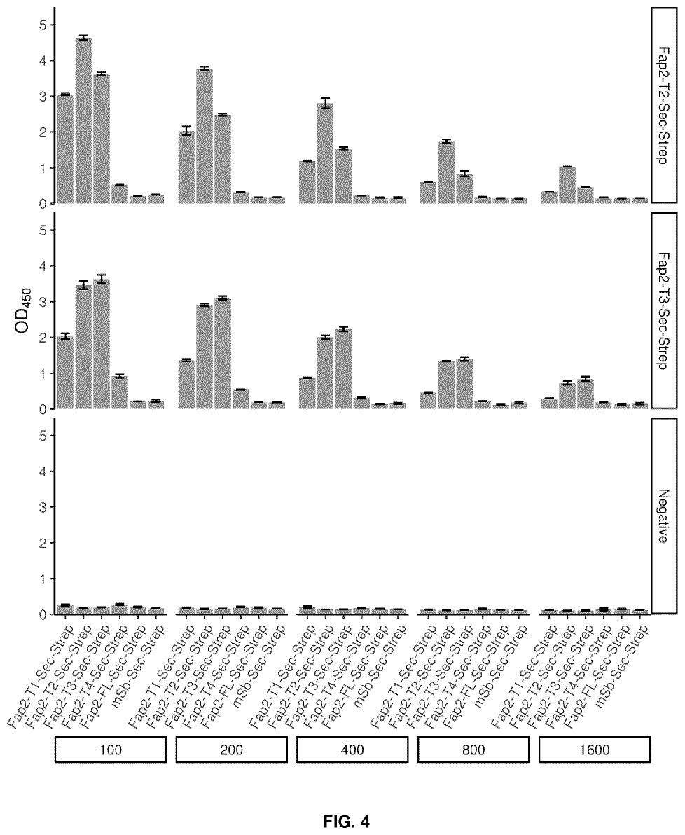

As shown in FIG. 4, secreted Fap2 truncation vaccine plasma pools similarly exhibited reactivity to Fap2-T2 and Fap2-T3 in addition to Fap2-FL. To examine for Fap2-specific antibodies, HEK293T/17 cells were transfected with Fap2-T2-Sec-Strep or Fap2-T3-Sec-Strep plasmid DNA using TransIT-LT1. At ˜48 hours following transfection, transfectant media was collected. Negative control (untransfected) media was also collected. Transfectant or negative control media was then used to coat pre-blocked streptavidin-coated 96-well plates (100 ul of media/well) after washing the plates 3× with ELISA wash buffer. Plates were incubated for 2 hours at 4 C to allow for antigen binding. Following coating, plates were washed 3× with ELISA wash buffer and 100 ul of diluted plasma samples were added to wells in duplicate. Plates were incubated at 4 C overnight. Following overnight incubation, the plates were washed 3× with ELISA wash buffer, and 100 ul of HRP-conjugated goat anti-mouse IgG antibody (1/2000 dilution) was added per well and allowed to incubate at RT for 2 hours. Plates were washed 3× with ELISA wash buffer, and 100 ul of TMB substrate solution was added per well. Reactions were allowed to develop for ˜8 mins, then 100 ul of ELISA stop solution was added and OD450 was measured. Each plasma sample was assayed in duplicate. Facets represent dilutions of plasma (100-fold, 200-fold, 400-fold, 800-fold, or 1600-fold). Bars represent the mean OD450, and error bars represent the standard error of the mean.

### Example 5.0

### Development of Further Constructs for Improved Immunogenicity

To further improve the immunogenicity of the Fap2 antigens, a series of constructs were designed that provide increased antigen valency. These constructs contain a N-terminal secretion signal (an lg kappa signal peptide) that facilitates antigen secretion, a Fap2-antigenic domain (T1, T2, T3, T4, or “Full Length” [FL] from F. nucleatum 7/1), and a C-terminal transmembrane domain or multimerization domain (i.e. a self-assembling domain) that increases antigen valency. Eight such domains were chosen: two transmembrane anchors, derived from PDGFR and B7-1, respectively; and six self-assembling domains for the creation of protein nanoparticles, Foldon, Ferritin, E2p, mi3, AP205, and IMX313.

To further improve the immunogenicity of the Fap2 antigens, minimal antigens that remove the predicted N-terminal disordered region were created. These antigens, T2ΔT1 and T3ΔT1, correspond to amino acid residues 372-1080 and 372-1627, respectively, of Fap2 from F. nucleatum 7/1 (3799 aa in length). In addition, these antigen constructs contain N-terminal secretion signals (an Ig kappa signal peptide) that facilitate antigen secretion, and various C-terminal domains that allow for either soluble secretion of monomeric antigen, or increased antigen valency. Nine C-terminal domains were used: A strep-tag; two transmembrane anchors, derived from PDGFR and B7-1, respectively; and six self-assembling domains for the creation of protein nanoparticles, Foldon, Ferritin, E2p, mi3, AP205, and IMX313. In some embodiments, each of the foregoing C-terminal domains may further be provided with a strep-tag, for example at the C-terminal portion of the transmembrane anchor or the self-assembling domain.

TABLE 2
Fap2 antigen targets with increased valency and/or increased immunogenicity.

| | | | Amino Acid |
|---|---|---|---|
| | | | Positions of |
| | | Amino Acid | Corresponding |
| Construct | DNA Sequence | Sequence | Features |
| FL-Fap2 with Ig kappa | SEQ ID NO: 11 | SEQ ID NO: 46 | 1-21: Ig kappa signal |
| signal peptide and AP205 | | | peptide |
| self-assembling domain | | | 22-3474: FL-Fap2 |
| | | | domain |
| | | | 3487-3617: AP205 |
| | | | domain |
| FL-Fap2 with Ig kappa | SEQ ID NO: 12 | SEQ ID NO: 47 | 1-21: Ig kappa signal |
| signal peptide, strep-tag, | | | peptide |
| and B7-1 transmembrane | | | 22-3474: FL-Fap2 |
| anchor | | | domain |
| | | | 3479-3548: B7 |
| | | | transmembrane |
| | | | domain |
| | | | 3552-3561: Strep-tag |
| FL-Fap2 with Ig kappa | SEQ ID NO: 13 | SEQ ID NO: 48 | 1-21: Ig kappa signal |
| signal peptide and E2p | | | peptide |
| self-assembling domain | | | 22-3474: FL-Fap2 |
| | | | domain |
| | | | 3487-3740: E2p |
| | | | domain |
| FL-Fap2 with Ig kappa | SEQ ID NO: 14 | SEQ ID NO: 49 | 1-21: Ig kappa signal |
| signal peptide and Ferritin | | | peptide |
| self-assembling domain | | | 22-3474: FL-Fap2 |
| | | | domain |
| | | | 3487-3659: Ferritin |
| | | | domain |
| FL-Fap2 with Ig kappa | SEQ ID NO: 15 | SEQ ID NO: 50 | 1-21: Ig kappa signal |
| signal peptide and Foldon | | | peptide |
| self-assembling domain | | | 22-3474: FL-Fap2 |
| | | | domain |
| | | | 3487-3513: Foldon |
| | | | domain |
| FL-Fap2 with Ig kappa | SEQ ID NO: 16 | SEQ ID NO: 51 | 1-21: Ig kappa signal |
| signal peptide and mi3 | | | peptide |
| self-assembling domain | | | 22-3474: FL-Fap2 |
| | | | domain |
| | | | 3487-3691: mi3 |
| | | | domain |
| FL-Fap2 with Ig kappa | SEQ ID NO: 17 | SEQ ID NO: 52 | 1-21: Ig kappa signal |
| signal peptide, strep-tag, | | | peptide |
| and PDGFR | | | 22-3474: FL-Fap2 |
| transmembrane anchor | | | domain |
| | | | 3479-3527: PDGFR |
| | | | transmembrane |
| | | | domain |
| | | | 3531-3540: Strep-tag |
| T1 antigen of Fap2 with Ig | SEQ ID NO: 18 | SEQ ID NO: 53 | 1-21: Ig kappa signal |
| kappa signal peptide and | | | peptide |
| AP205 self-assembling | | | 22-350: T1-Fap2 |
| domain | | | domain |
| | | | 363-493: AP205 |
| | | | domain |
| T1 antigen of Fap2 with Ig | SEQ ID NO: 19 | SEQ ID NO: 54 | 1-21: Ig kappa signal |
| kappa signal peptide, | | | peptide |
| strep-tag, and B7-1 | | | 22-350: T1-Fap2 |
| transmembrane anchor | | | domain |
| | | | 355-424: B7 |
| | | | transmembrane |
| | | | domain |
| | | | 428-437: Strep-tag |
| T1 antigen of Fap2 with Ig | SEQ ID NO: 20 | SEQ ID NO: 55 | 1-21: Ig kappa signal |
| kappa signal peptide and | | | peptide |
| E2p self-assembling | | | 22-350: T1-Fap2 |
| domain | | | domain |
| | | | 363-616: E2p domain |
| T1 antigenic domain of | SEQ ID NO: 21 | SEQ ID NO: 56 | 1-21: Ig kappa signal |
| Fap2 with Ig kappa signal | | | peptide |
| peptide and Ferritin self- | | | 22-350: T1-Fap2 |
| assembling domain | | | domain |
| | | | 363-535: Ferritin |
| | | | domain |
| T1 antigen of Fap2 with Ig | SEQ ID NO: 22 | SEQ ID NO: 57 | 1-21: Ig kappa signal |
| kappa signal peptide and | | | peptide |
| Foldon self-assembling | | | 22-350: T1-Fap2 |
| domain | | | domain |
| | | | 363-389: Foldon |
| | | | domain |
| T1 antigen of Fap2 with Ig | SEQ ID NO: 23 | SEQ ID NO: 58 | 1-21: Ig kappa signal |
| kappa signal peptide and | | | peptide |
| mi3 self-assembling | | | 22-350: T1-Fap2 |
| domain | | | domain |
| | | | 363-567: mi3 domain |
| T1 antigen of Fap2 with Ig | SEQ ID NO: 24 | SEQ ID NO: 59 | 1-21: Ig kappa signal |
| kappa signal peptide, | | | peptide |
| strep-tag, and PDGFR | | | 22-350: T1-Fap2 |
| transmembrane anchor | | | domain |
| | | | 355-403: PDGFR |
| | | | transmembrane |
| | | | domain |
| | | | 407-416: Strep-tag |
| T2 antigen of Fap2 with Ig | SEQ ID NO: 25 | SEQ ID NO: 60 | 1-21: Ig kappa signal |
| kappa signal peptide and | | | 22-1059: T2-Fap2 |
| AP205 self-assembling | | | domain |
| domain | | | 1072-1202: AP205 |
| | | | domain |
| T2 antigen of Fap2 with Ig | SEQ ID NO: 26 | SEQ ID NO: 61 | 1-21: Ig kappa signal |
| kappa signal peptide, | | | peptide |
| strep-tag, and B7-1 | | | 22-1059: T2-Fap2 |
| transmembrane anchor | | | domain |
| | | | 1064-1133: B7 |
| | | | transmembrane |
| | | | domain |
| | | | 1137-1146: Strep-tag |
| T2 antigen of Fap2 with Ig | SEQ ID NO: 27 | SEQ ID NO: 62 | 1-21: Ig kappa signal |
| kappa signal peptide and | | | peptide |
| E2p self-assembling | | | 22-1059: T2-Fap2 |
| domain | | | domain |
| | | | 1072-1325: E2p |
| | | | domain |
| T2 antigen of Fap2 with Ig | SEQ ID NO: 28 | SEQ ID NO: 63 | 1-21: Ig kappa signal |
| kappa signal peptide and | | | peptide |
| Ferritin self-assembling | | | 22-1059: T2-Fap2 |
| domain | | | domain |
| | | | 1072-1244: Ferritin |
| | | | domain |
| T2 antigen of Fap2 with Ig | SEQ ID NO: 29 | SEQ ID NO: 64 | 1-21: Ig kappa signal |
| kappa signal peptide and | | | peptide |
| Foldon self-assembling | | | 22-1059: T2-Fap2 |
| domain | | | domain |
| | | | 1072-1098: Foldon |
| | | | domain |
| T2 antigen of Fap2 with Ig | SEQ ID NO: 30 | SEQ ID NO: 65 | 1-21: Ig kappa signal |
| kappa signal peptide and | | | peptide |
| mi3 self-assembling | | | 22-1059: T2-Fap2 |
| domain | | | domain |
| | | | 1072-1276: mi3 |
| | | | domain |
| T2 antigen of Fap2 with Ig | SEQ ID NO: 31 | SEQ ID NO: 66 | 1-21: Ig kappa signal |
| kappa signal peptide, | | | peptide |
| strep-tag, and PDGFR | | | 22-1059: T2-Fap2 |
| transmembrane anchor | | | domain |
| | | | 1064-1112: PDGFR |
| | | | transmembrane |
| | | | domain |
| | | | 1116-1125: Strep-tag |
| T3 antigen of Fap2 with Ig | SEQ ID NO: 32 | SEQ ID NO: 67 | 1-21: Ig kappa signal |
| kappa signal peptide and | | | 22-1606: T3-Fap2 |
| AP205 self-assembling | | | domain |
| domain | | | 1619-1749: AP205 |
| | | | domain |
| T3 antigen of Fap2 with Ig | SEQ ID NO: 33 | SEQ ID NO: 68 | 1-21: Ig kappa signal |
| kappa signal peptide, | | | peptide |
| strep-tag, and B7-1 | | | 22-1606: T3-Fap2 |
| transmembrane anchor | | | domain |
| | | | 1611-1680: B7 |
| | | | transmembrane |
| | | | domain |
| | | | 1684-1693: Strep-tag |
| T3 antigen of Fap2 with Ig | SEQ ID NO: 34 | SEQ ID NO: 69 | 1-21: Ig kappa signal |
| kappa signal peptide and | | | peptide |
| E2p self-assembling | | | 22-1606: T3-Fap2 |
| domain | | | domain |
| | | | 1619-1872: E2p |
| | | | domain |
| T3 antigen of Fap2 with Ig | SEQ ID NO: 35 | SEQ ID NO: 70 | 1-21: Ig kappa signal |
| kappa signal peptide and | | | peptide |
| Ferritin self-assembling | | | 22-1606: T3-Fap2 |
| domain | | | domain |
| | | | 1619-1791: Ferritin |
| | | | domain |
| T3 antigen of Fap2 with Ig | SEQ ID NO: 36 | SEQ ID NO: 71 | 1-21: Ig kappa signal |
| kappa signal peptide and | | | peptide |
| Foldon self-assembling | | | 22-1606: T3-Fap2 |
| domain | | | domain |
| | | | 1619-1645: Foldon |
| | | | domain |
| T3 antigen of Fap2 with Ig | SEQ ID NO: 37 | SEQ ID NO: 72 | 1-21: Ig kappa signal |
| kappa signal peptide and | | | peptide |
| mi3 self-assembling | | | 22-1606: T3-Fap2 |
| domain | | | domain |
| | | | 1619-1823: mi3 |
| | | | domain |
| T3 antigen of Fap2 with Ig | SEQ ID NO: 38 | SEQ ID NO: 73 | 1-21: Ig kappa signal |
| kappa signal peptide, | | | peptide |
| strep-tag, and PDGFR | | | 22-1606: T3-Fap2 |
| transmembrane anchor | | | domain |
| | | | 1611-1659: PDGFR |
| | | | transmembrane |
| | | | domain |
| | | | 1663-1672: Strep-tag |
| T4 antigen of Fap2 with Ig | SEQ ID NO: 39 | SEQ ID NO: 74 | 1-21: Ig kappa signal |
| kappa signal peptide and | | | 22-2252: T4-Fap2 |
| AP205 self-assembling | | | domain |
| domain | | | 2266-2396: AP205 |
| | | | domain |
| T4 antigen of Fap2 with Ig | SEQ ID NO: 40 | SEQ ID NO: 75 | 1-21: Ig kappa signal |
| kappa signal peptide, | | | peptide |
| strep-tag, and B7-1 | | | 22-2252: T4-Fap2 |
| transmembrane anchor | | | domain |
| | | | 2258-2327: B7 |
| | | | transmembrane |
| | | | domain |
| | | | 2331-2340: Strep-tag |
| T4 antigen of Fap2 with Ig | SEQ ID NO: 41 | SEQ ID NO: 76 | 1-21: Ig kappa signal |
| kappa signal peptide and | | | peptide |
| E2p self-assembling | | | 22-2252: T4-Fap2 |
| domain | | | domain |
| | | | 2266-2519: E2p |
| | | | domain |
| T4 antigen of Fap2 with Ig | SEQ ID NO: 42 | SEQ ID NO: 77 | 1-21: Ig kappa signal |
| kappa signal peptide and | | | peptide |
| Ferritin self-assembling | | | 22-2252: T4-Fap2 |
| domain | | | domain |
| | | | 2266-2438: Ferritin |
| | | | domain |
| T4 antigen of Fap2 with Ig | SEQ ID NO: 43 | SEQ ID NO: 78 | 1-21: Ig kappa signal |
| kappa signal peptide and | | | peptide |
| Foldon self-assembling | | | 22-2252: T4-Fap2 |
| domain | | | domain |
| | | | 2266-2292: Foldon |
| | | | domain |
| T4 antigen of Fap2 with Ig | SEQ ID NO: 44 | SEQ ID NO: 79 | 1-21: Ig kappa signal |
| kappa signal peptide and | | | peptide |
| mi3 self-assembling | | | 22-2252: T4-Fap2 |
| domain | | | domain |
| | | | 2266-2470: mi3 |
| | | | domain |
| T4 antigen of Fap2 with Ig | SEQ ID NO: 45 | SEQ ID NO: 80 | 1-21: Ig kappa signal |
| kappa signal peptide, | | | peptide |
| strep-tag, and PDGFR | | | 22-2252: T4-Fap2 |
| transmembrane anchor | | | domain |
| | | | 2258-2306: PDGFR |
| | | | transmembrane |
| | | | domain |
| | | | 2310-2319: Strep-tag |
| T2ΔT1 antigen of Fap2 | SEQ ID NO: 81 | SEQ ID NO: 97 | 1-21: Ig kappa signal |
| with Ig kappa signal | | | 23-730: T2ΔT1-Fap2 |
| peptide and strep-tag | | | domain |
| | | | 734-743: Strep-tag |
| T2ΔT1 antigen of Fap2 | SEQ ID NO: 82 | SEQ ID NO: 98 | 1-21: Ig kappa signal |
| with Ig kappa signal | | | 23-730: T2ΔT1-Fap2 |
| peptide and AP205 self- | | | domain |
| assembling domain | | | 743-873: AP205 |
| | | | domain |
| T2ΔT1 antigen of Fap2 | SEQ ID NO: 83 | SEQ ID NO: 99 | 1-21: Ig kappa signal |
| with Ig kappa signal | | | peptide |
| peptide, strep-tag, and B7- | | | 23-730: T2ΔT1-Fap2 |
| 1 transmembrane anchor | | | domain |
| | | | 735-804: B7 |
| | | | transmembrane |
| | | | domain |
| | | | 808-817: Strep-tag |
| T2ΔT1 antigen of Fap2 | SEQ ID NO: 84 | SEQ ID NO: 100 | 1-21: Ig kappa signal |
| with Ig kappa signal | | | 23-730: T2ΔT1-Fap2 |
| peptide and E2p self- | | | domain |
| assembling domain | | | 743-996: E2p domain |
| T2ΔT1 antigen of Fap2 | SEQ ID NO: 85 | SEQ ID NO: 101 | 1-21: Ig kappa signal |
| with Ig kappa signal | | | 23-730: T2ΔT1-Fap2 |
| peptide and Ferritin self- | | | domain |
| assembling domain | | | 743-915: Ferritin |
| | | | domain |
| T2ΔT1 antigen of Fap2 | SEQ ID NO: 86 | SEQ ID NO: 102 | 1-21: Ig kappa signal |
| with Ig kappa signal | | | 23-730: T2ΔT1-Fap2 |
| peptide and Foldon self- | | | domain |
| assembling domain | | | 743-769: Foldon |
| | | | domain |
| T2ΔT1 antigen of Fap2 | SEQ ID NO: 87 | SEQ ID NO: 103 | 1-21: Ig kappa signal |
| with Ig kappa signal | | | 23-730: T2ΔT1-Fap2 |
| peptide and mi3 self- | | | domain |
| assembling domain | | | 743-947: mi3 domain |
| T2ΔT1 antigen of Fap2 | SEQ ID NO: 88 | SEQ ID NO: 104 | 1-21: Ig kappa signal |
| with Ig kappa signal | | | 23-730: T2ΔT1-Fap2 |
| peptide, strep-tag, and | | | domain |
| PDGFR transmembrane | | | 735-783: PDGFR |
| anchor | | | transmembrane |
| | | | domain |
| | | | 787-796: Strep-tag |
| T3ΔT1 antigen of Fap2 | SEQ ID NO: 89 | SEQ ID NO: 105 | 1-21: Ig kappa signal |
| with Ig kappa signal | | | 23-1277: T3ΔT1-Fap2 |
| peptide and strep-tag | | | domain |
| | | | 1281-1290: Strep-tag |
| T3ΔT1 antigen of Fap2 | SEQ ID NO: 90 | SEQ ID NO: 106 | 1-21: Ig kappa signal |
| with Ig kappa signal | | | 23-1277: T3ΔT1-Fap2 |
| peptide and AP205 self- | | | domain |
| assembling domain | | | 1290-1420: AP205 |
| | | | domain |
| T3ΔT1 antigen of Fap2 | SEQ ID NO: 91 | SEQ ID NO: 107 | 1-21: Ig kappa signal |
| with Ig kappa signal | | | peptide |
| peptide, strep-tag, and B7- | | | 23-1277: T3ΔT1-Fap2 |
| 1 transmembrane anchor | | | domain |
| | | | 1282-1351: B7 |
| | | | transmembrane |
| | | | domain |
| | | | 1355-1364: Strep-tag |
| T3ΔT1 antigen of Fap2 | SEQ ID NO: 92 | SEQ ID NO: 108 | 1-21: Ig kappa signal |
| with Ig kappa signal | | | 23-1277: T3ΔT1-Fap2 |
| peptide and E2p self- | | | domain |
| assembling domain | | | 1290-1543: E2p |
| | | | domain |
| T3ΔT1 antigen of Fap2 | SEQ ID NO: 93 | SEQ ID NO: 109 | 1-21: Ig kappa signal |
| with Ig kappa signal | | | 23-1277: T3ΔT1-Fap2 |
| peptide and Ferritin self- | | | domain |
| assembling domain | | | 1290-1462: Ferritin |
| | | | domain |
| T3ΔT1 antigen of Fap2 | SEQ ID NO: 94 | SEQ ID NO: 110 | 1-21: Ig kappa signal |
| with Ig kappa signal | | | 23-1277: T3ΔT1-Fap2 |
| peptide and Foldon self- | | | domain |
| assembling domain | | | 1290-1316: Foldon |
| | | | domain |
| T3ΔT1 antigen of Fap2 | SEQ ID NO: 95 | SEQ ID NO: 111 | 1-21: Ig kappa signal |
| with Ig kappa signal | | | 23-1277: T3ΔT1-Fap2 |
| peptide and mi3 self- | | | domain |
| assembling domain | | | 1290-1494: mi3 |
| | | | domain |
| T3ΔT1 antigen of Fap2 | SEQ ID NO: 96 | SEQ ID NO: 112 | 1-21: Ig kappa signal |
| with Ig kappa signal | | | 23-1277: T3ΔT1-Fap2 |
| peptide, strep-tag, and | | | domain |
| PDGFR transmembrane | | | 1282-1330: PDGFR |
| anchor | | | transmembrane |
| | | | domain |
| | | | 1334-1343: Strep-tag |
| FL-Fap2 with Ig kappa | SEQ ID NO: 113 | SEQ ID NO: 120 | 1-21: Ig kappa signal |
| signal peptide and IMX313 | | | 22-3474: FL-Fap2 |
| multimerization domain | | | domain |
| | | | 3487-3541: IMX313 |
| | | | domain |
| T1 antigen domain of Fap | SEQ ID NO: 114 | SEQ ID NO: 121 | 1-21: Ig kappa signal |
| 2 with Ig kappa signal | | | 22-350: T1-Fap2 |
| peptide and IMX313 | | | domain |
| multimerization domain | | | 363-417: IMX313 |
| | | | domain |
| T2 antigen domain of Fap | SEQ ID NO: 115 | SEQ ID NO: 122 | 1-21: Ig kappa signal |
| 2 with Ig kappa signal | | | 22-1059: T2-Fap2 |
| peptide and IMX313 | | | domain |
| multimerization domain | | | 1072-1126: IMX313 |
| | | | domain |
| T2ΔT1 antigen domain of | SEQ ID NO: 116 | SEQ ID NO: 123 | 1-21: Ig kappa signal |
| Fap 2 with Ig kappa signal | | | 23-730: T2ΔT1-Fap2 |
| peptide and IMX313 | | | domain |
| multimerization domain | | | 743-797: IMX313 |
| | | | domain |
| T3 antigen domain of Fap | SEQ ID NO: 117 | SEQ ID NO: 124 | 1-21: Ig kappa signal |
| 2 with Ig kappa signal | | | 22-1606: T3-Fap2 |
| peptide and IMX313 | | | domain |
| multimerization domain | | | 1619-1673: IMX313 |
| | | | domain |
| T3ΔT1 antigen domain of | SEQ ID NO: 118 | SEQ ID NO: 125 | 1-21: Ig kappa signal |
| Fap 2 with Ig kappa signal | | | 23-1277: T3ΔT1-Fap2 |
| peptide and IMX313 | | | domain |
| multimerization domain | | | 1290-1344: IMX313 |
| | | | domain |
| T4 antigen domain of Fap | SEQ ID NO: 119 | SEQ ID NO: 126 | 1-21: Ig kappa signal |
| 2 with Ig kappa signal | | | 22-2252: T4-Fap2 |
| peptide and IMX313 | | | domain |
| multimerization domain | | | 2266-2320: IMX313 |
| | | | domain |

### Example 6.0

### Expression of FLAG-Tagged Transmembrane Displayed Fap2 Truncation pDNA Transfectants

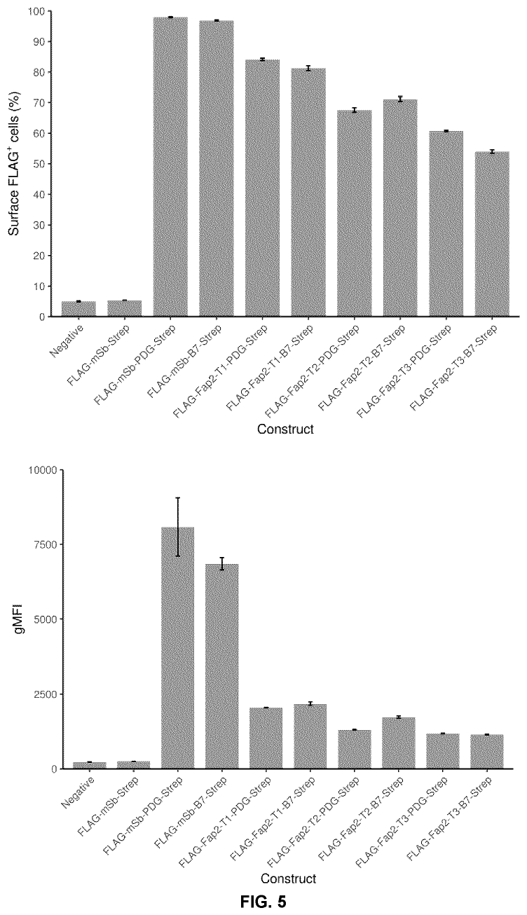

As shown in FIG. 5, flow cytometry was used to detect surface FLAG expression in FLAG-tagged transmembrane displayed Fap2 truncation pDNA transfectants. For this example, nine different constructs were prepared as follows. To prepare the FLAG-tagged variants, a FLAG tag sequence was inserted between the signal peptide and the coding sequence of the Fap2 variant as follows: in the DNA sequence between the nucleotides at positions 63 and 64,

GACTACAAAGACGATGACGACAAGGGCGGAGGCTCT (SEQ ID NO:4558), and in the amino acid sequence between residues 21 and 22, DYKDDDDKGGGS (SEQ ID NO: 4559).
FLAG-mSb-Strep—Ig kappa signal peptide preceding an N-terminal FLAG-tagged construct bearing the mSb reporter a C-terminal Strep tag;
FLAG-mSb-PDG-Strep—Ig kappa signal peptide preceding an N-terminal FLAG-tagged construct bearing the mSb reporter with a PDGFR transmembrane anchor and a C-terminal Strep tag;
FLAG-mSb-B7-Strep—Ig kappa signal peptide preceding an N-terminal FLAG-tagged construct bearing the mSb reporter with a B7-1 transmembrane anchor and a C-terminal Strep tag;
FLAG-Fap2-T1-PDG-Strep—Ig kappa signal peptide preceding an N-terminal FLAG-tagged construct bearing the Fap2 T1 domain with a PDGFR transmembrane anchor and a C-terminal Strep tag (SEQ ID NO:24/SEQ ID NO: 59 with FLAG-tag);
FLAG-Fap2-T1-B7-Strep—Ig kappa signal peptide preceding an N-terminal FLAG-tagged construct bearing the Fap2 T1 domain with a B7-1 transmembrane anchor and a C-terminal Strep tag (SEQ ID NO: 19/SEQ ID NO: 54 with FLAG-tag);
FLAG-Fap2-T2-PDG-Strep—Ig kappa signal peptide preceding an N-terminal FLAG-tagged construct bearing the Fap2 T2 domain with a PDGFR transmembrane anchor and a C-terminal Strep tag (SEQ ID NO:31/SEQ ID NO: 66 with FLAG-tag);
FLAG-Fap2-T2-B7-Strep—Ig kappa signal peptide preceding an N-terminal FLAG-tagged construct bearing the Fap2 T2 domain with a B7-1 transmembrane anchor and a C-terminal Strep tag (SEQ ID NO:26/SEQ ID NO:61 with FLAG-tag);
FLAG-Fap2-T3-PDG-Strep—Ig kappa signal peptide preceding an N-terminal FLAG-tagged construct bearing the Fap2 T3 domain with a PDGFR transmembrane anchor and a C-terminal Strep tag (SEQ ID NO:38/SEQ ID NO: 73 with FLAG-tag);
FLAG-Fap2-T3-B7-Strep—Ig kappa signal peptide preceding an N-terminal FLAG-tagged construct bearing the Fap2 T3 domain with a B7-1 transmembrane anchor and a C-terminal Strep tag (SEQ ID NO:33/SEQ ID NO:68 with FLAG-tag);
   An unstained negative control was also evaluated.

HEK293T/17 cells were plated at 2.5ml/well (540,000 cells/ml) in 6-well plates ˜19 hours prior to transfection. Cells were transfected with 2.5 ug of plasmid DNA using TransIT-LT1. Cells were harvested for analysis at ˜24 hours following transfection. Cells were stained with FITC-conjugated anti-FLAG for 30 mins, and then stained with DAPI. Cells were analyzed on a BD Fortessa flow cytometer. Resulting events were gated to isolate singlet live cells, and FLAG-positivity was determined relative to an unstained control. All transfections were performed in duplicate. Bars represent the mean FLAG-positivity (top) or gMFI (geometric mean fluorescent intensity) (bottom), and error bars represent the standard error of the mean.

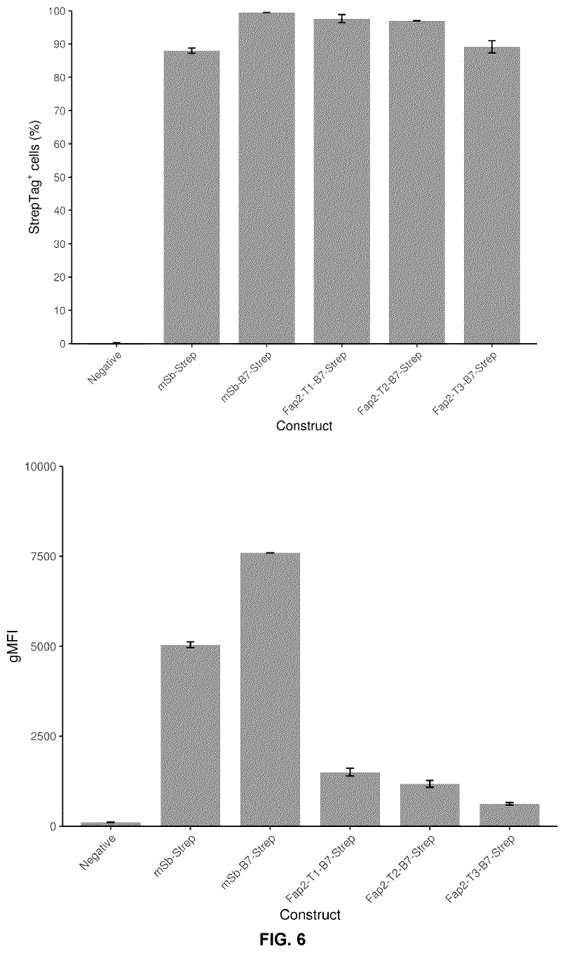

As shown in FIG. 6, flow cytometry was also used to detect expression of StrepTagII in the transmembrane displayed Fap2 truncation pDNA transfectants. The B7 transmembrane domain was selected as an exemplary representative transmembrane anchor for this example. HEK293T/17 cells were plated at 2.5 ml/well (540,000 cells/ml) in 6-well plates ˜19 hours prior to transfection. Cells were transfected with 2.5 ug of plasmid DNA using TransIT-LT1. Cells were harvested for analysis at ˜24 hours following transfection. Cells were stained with Fixable Viability Dye eFluor780 for 30 mins, fixed with 4% formaldehyde for 15 mins, permeabilized with cell permeablization buffer for 10 mins, and then stained with FITC-conjugated mouse anti-StrepTagII antibody for 30 mins. Cells were analyzed on a BD Fortessa flow cytometer. Resulting events were gated to isolate singlet live cells, and StrepTagII-positivity was determined relative to an unstained control. All transfections were performed in duplicate. Bars represent the mean StrepTagII-positivity (top) or gMFI (bottom), and error bars represent the standard error of the mean.

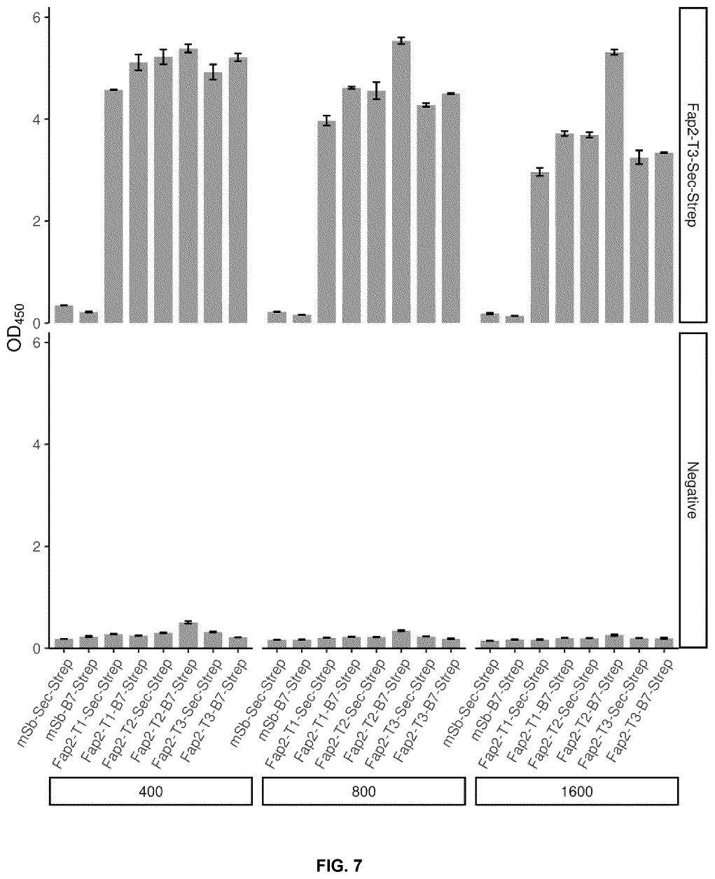

The reactivity of the transmembrane-displayed Fap2 truncation vaccine serum pools to Fap2-T3 was tested and compared with secreted Fap2 truncation vaccine serum pools to show that both types of construct result in the generation of Fap2-specific antibodies. As shown in FIG. 7, to examine for Fap2-specific antibodies, HEK293T/17 cells were transfected with Fap2-T3-Sec-Strep plasmid DNA using TransIT-LT1. At ˜52 hours following transfection, transfectant media was collected. Negative control (mock-transfected) media was also collected. Transfectant or negative control media was then used to coat pre-blocked streptavidin-coated 96-well plates (100 ul of media/well) after washing the plates 3× with ELISA wash buffer. Plates were incubated for 2 hours at 4 C to allow for antigen binding. Following coating, plates were washed 3× with ELISA wash buffer and 100 ul of diluted serum samples were added to wells in duplicate. Plates were incubated at 4 C overnight. Following overnight incubation, the plates were washed 3× with ELISA wash buffer, and 100 ul of HRP-conjugated donkey anti-mouse IgG antibody (1/5000 dilution) was added per well and allowed to incubate for 2 hours. Plates were washed 3× with ELISA wash buffer, and 100 ul of TMB substrate solution was added per well. Reactions were allowed to develop for ˜4 mins, then 100 ul of ELISA stop solution was added and OD450 was measured. Each serum sample was assayed in duplicate. Facets represent dilutions of serum (400-fold, 800-fold, or 1600-fold). Bars represent the mean OD450, and error bars represent the standard error of the mean.

### Example 7.0

### Reactivity of B-cell Hybridoma Media Samples to Fap2 Antigens

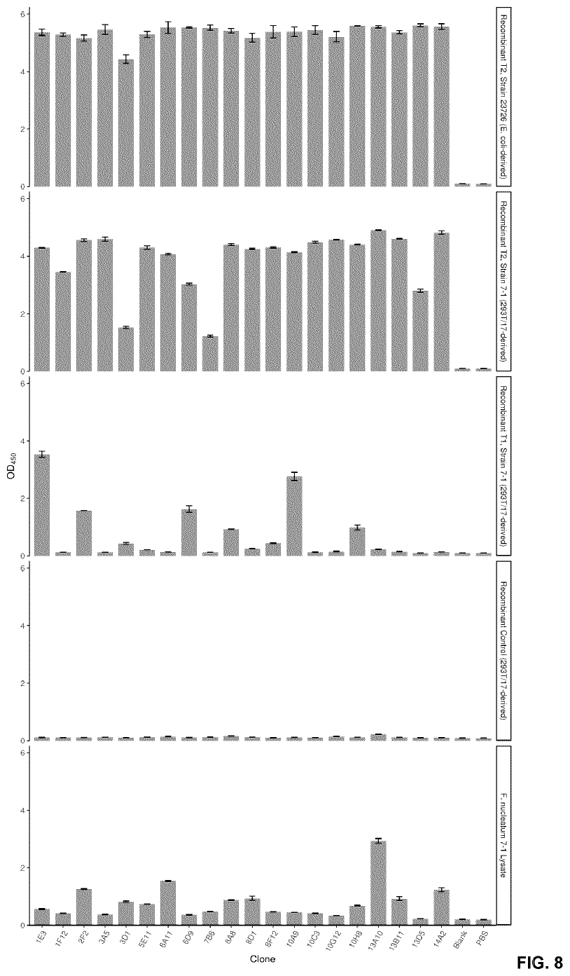

As shown in FIG. 8, the reactivity of B-cell hybridoma media samples to various Fap2 antigen sources was examined. Mice were vaccinated with E. coli-derived protein of the Fap2 T2 segment from F. nucleatum strain 23726 and hybridoma cell pools were created. To examine for Fap2-specific antibodies in hybridoma clone pool media samples (identified along the x-axis), plates were coated with various forms of Fap2 antigen and hybridoma pool media samples containing secreted antibodies were assayed via ELISA. The amino acid sequence of full length Fap2 from F. Nucleatum strain 23726 is provided in SEQ ID NO:4560, and the amino acid sequence of the Fap2 T2 segment from F. Nucleatum strain 23726 is provided in SEQ ID NO:4561. In this example, the Fap2 T2 segment included an N-terminal His-tag added to facilitate purification (SEQ ID NO:4562).

Top Panel: E. coli-derived protein of the Fap2 T2 segment from F. nucleatum strain 23726 was coated onto Maxisorp plates at 10 ug/ml using 100 ul/well and incubated overnight. Plates were washed 3× with ELISA wash buffer and blocked. Following blocking, plates were washed 3× with ELISA wash buffer and 50 ul of diluted hybridoma pool media samples were added to wells in duplicate. Plates were incubated at 4 C overnight. Following overnight incubation, the plates were washed 3× with ELISA wash buffer, and 100 ul of HRP-conjugated donkey anti-mouse IgG antibody (1/5000 dilution) was added per well and allowed to incubate at RT for 2 hours. Plates were washed 3× with ELISA wash buffer, and 100 ul of TMB substrate solution was added per well. Reactions were allowed to develop for ˜2.5 mins, then 100 ul of ELISA stop solution was added and OD450 was measured. Each media sample was assayed in duplicate. Bars represent the mean OD450, and error bars represent the standard error of the mean.

Middle Three Panels: HEK293T/17 cells were transfected with Fap2-T1-Sec-Strep or Fap2-T2-Sec-Strep plasmid DNA from F. nucleatum strain 7-1 using TransIT-LT1. At ˜48 hours following transfection, transfectant media was collected. Negative control (mock-transfected) media was also collected. Transfectant or negative control media was then used to coat pre-blocked streptavidin-coated 96-well plates (100 ul of media/well) after washing the plates 3× with ELISA wash buffer. Plates were incubated for 3 hours at 4 C to allow for antigen binding. Following coating, plates were washed 3× with ELISA wash buffer and 50 ul of diluted hybridoma pool media samples were added to wells in duplicate. Plates were incubated at 4 C overnight. Following overnight incubation, the plates were washed 3× with ELISA wash buffer, and 100 ul of HRP-conjugated donkey anti-mouse IgG antibody (1/5000 dilution) was added per well and allowed to incubate at RT for 2 hours. Plates were washed 3× with ELISA wash buffer, and 100 ul of TMB substrate solution was added per well. Reactions were allowed to develop for ˜2.5 mins, then 100 ul of ELISA stop solution was added and OD450 was measured. Each media sample was assayed in duplicate. Bars represent the mean OD450, and error bars represent the standard error of the mean.

Bottom panel: F. nucleatum strain 7-1 lysate (extracted using B-PER Complete Reagent) was coated onto Maxisorp plates at 10 ug/ml using 100 ul/well and incubated overnight. Plates were washed 3× with ELISA wash buffer and blocked. Following blocking, plates were washed 3× with ELISA wash buffer and 50 ul of diluted hybridoma pool media samples were added to wells in duplicate. Plates were incubated at 4 C overnight. Following overnight incubation, the plates were washed 3× with ELISA wash buffer, and 100 ul of HRP-conjugated donkey anti-mouse IgG antibody (1/5000 dilution) was added per well and allowed to incubate at RT for 2 hours. Plates were washed 3× with ELISA wash buffer, and 100 ul of TMB substrate solution was added per well. Reactions were allowed to develop for ˜20 mins, then 100 ul of ELISA stop solution was added and OD450 was measured. Each media sample was assayed in duplicate. Bars represent the mean OD450, and error bars represent the standard error of the mean.

### Example 8.0

### Prediction of Additional Possible Fap2 Epitopes

BepiPred-2.0 was used to predict B cell epitope probabilities for Fap2 from each of the nine Fusobacterium nucleatum subsp. Input for the prediction is the protein sequence, output of the prediction is a probability, per amino acid, that that amino acid is part of a B cell epitope. Different Epitope Probability thresholds can be chosen, with different associated sensitivity and specificity. The inventors used three approaches to identify regions of increased epitope density and specific predicted epitopes.

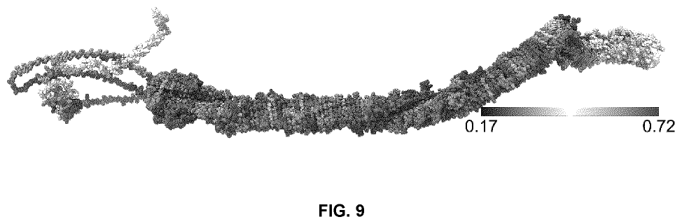

As shown in FIG. 9, first, for F. nucleatum 7-1, the inventors mapped the per-amino-acid Epitope Probability scores to a gradient to display on the predicted Fap2 structure. This shows the main body of Fap2 having the highest Epitope Probability scores.

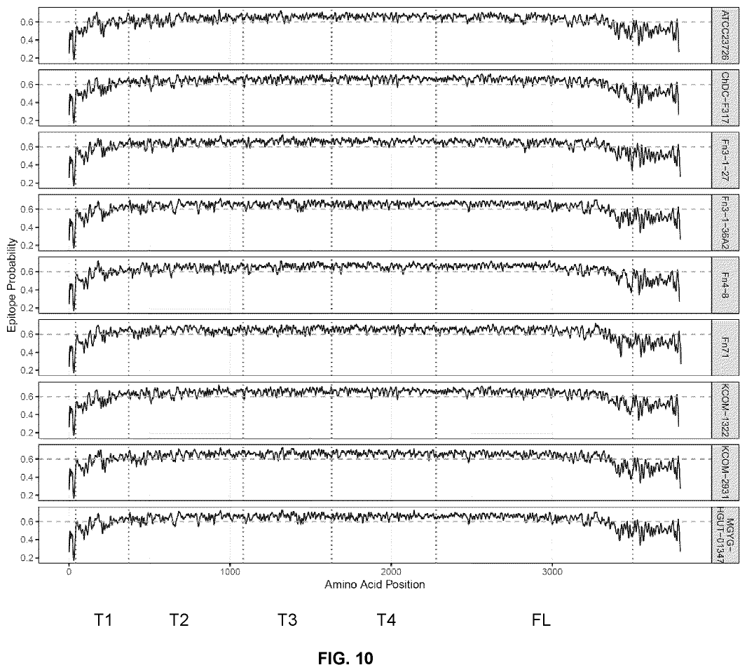

Next, as shown in FIG. 10, the inventors displayed the per-amino-acid Epitope Probability for each F. nucleatum subsp. in a plot. A horizontal dashed line is drawn at 0.6, which corresponds to a specificity of 95% and a sensitivity of 10%. Vertical dotted lines are drawn at the different truncation boundaries, and this shows how the truncations T1, T2, T3 and T4 tested above were selected to contain the regions with the highest probability of harbouring B cell epitopes.

The final approach was to start with an Epitope Probability threshold of 1.00, and slowly decrease this until the amino acids that fall above the threshold represent at least 10 contiguous linear epitopes that are 12aa long. The threshold was selected independently for each F. nucleatum subsp., though ended up being 0.68 for each. Table 3 summarizes the F. nucleatum subsp. that was the source of the Fap2 sequence, the threshold (the Epitope Probability threshold that was used), the epitope (the 12aa linear B cell epitope), and the starting position of the epitope (the position of the Fap2 reference protein where the epitope is found). Additionally, the inventors determined for each position along the predicted epitope the per-amino-acid Epitope Probabilities (data not shown).

TABLE 3
B-Cell Epitope Predictions for Various F. nucleatum subsp.

| F. nucleatum | | | Start | |
|---|---|---|---|---|
| subsp. | Threshold | Epitope | Pos. | SEQ ID NO: |
| Fn7/1 (i.e. | 0.68 | SLNSWKNANNSS | 173 | SEQ ID NO: 127 |
| F. nucleatum | | | | |
| 7/1) | | | | |
| Fn7/1 | 0.68 | LNSWKNANNSSN | 174 | SEQ ID NO: 128 |
| Fn7/1 | 0.68 | ETDTLINGSGAR | 718 | SEQ ID NO: 129 |
| Fn7/1 | 0.68 | TDTLINGSGARN | 719 | SEQ ID NO: 130 |
| Fn7/1 | 0.68 | DTLINGSGARNT | 720 | SEQ ID NO: 131 |
| Fn7/1 | 0.68 | TLINGSGARNTA | 721 | SEQ ID NO: 132 |
| Fn7/1 | 0.68 | LINGSGARNTAT | 722 | SEQ ID NO: 133 |
| Fn7/1 | 0.68 | KSSQANIESVVT | 1544 | SEQ ID NO: 134 |
| Fn7/1 | 0.68 | EESAGMYVENSS | 1772 | SEQ ID NO: 135 |
| Fn7/1 | 0.68 | ESAGMYVENSSA | 1773 | SEQ ID NO: 136 |
| Fn7/1 | 0.68 | SAGMYVENSSAT | 1774 | SEQ ID NO: 137 |
| Fn7/1 | 0.68 | AGMYVENSSATN | 1775 | SEQ ID NO: 138 |
| Fn7/1 | 0.68 | GMYVENSSATNK | 1776 | SEQ ID NO: 139 |
| Fn7/1 | 0.68 | MYVENSSATNKK | 1777 | SEQ ID NO: 140 |
| Fn7/1 | 0.68 | QKQINSKISSDP | 3266 | SEQ ID NO: 141 |
| ATCC23726 | 0.68 | DTLINSSGARNT | 715 | SEQ ID NO: 142 |
| ATCC23726 | 0.68 | TLINSSGARNTA | 716 | SEQ ID NO: 143 |
| ATCC23726 | 0.68 | LINSSGARNTAT | 717 | SEQ ID NO: 144 |
| ATCC23726 | 0.68 | NESTQANTQSEV | 1308 | SEQ ID NO: 145 |
| ATCC23726 | 0.68 | ESTQANTQSEVT | 1309 | SEQ ID NO: 146 |
| ATCC23726 | 0.68 | STQANTQSEVTN | 1310 | SEQ ID NO: 147 |
| ATCC23726 | 0.68 | TQANTQSEVTNS | 1311 | SEQ ID NO: 148 |
| ATCC23726 | 0.68 | EESVGMYSSSSL | 1715 | SEQ ID NO: 149 |
| ATCC23726 | 0.68 | ESVGMYSSSSLK | 1716 | SEQ ID NO: 150 |
| ATCC23726 | 0.68 | SVGMYSSSSLKA | 1717 | SEQ ID NO: 151 |
| ATCC23726 | 0.68 | TLDKNTSKLDYT | 2204 | SEQ ID NO: 152 |
| ATCC23726 | 0.68 | LDKNTSKLDYTL | 2205 | SEQ ID NO: 153 |
| ATCC23726 | 0.68 | DKNTSKLDYTLQ | 2206 | SEQ ID NO: 154 |
| ATCC23726 | 0.68 | KNTSKLDYTLQG | 2207 | SEQ ID NO: 155 |
| ATCC23726 | 0.68 | NTSKLDYTLQGT | 2208 | SEQ ID NO: 156 |
| ATCC23726 | 0.68 | TSKLDYTLQGTG | 2209 | SEQ ID NO: 157 |
| ATCC23726 | 0.68 | ENKGGQITSESG | 2559 | SEQ ID NO: 158 |
| ChDC-F317 | 0.68 | KEEESVGMYSSS | 1712 | SEQ ID NO: 159 |
| ChDC-F317 | 0.68 | EEESVGMYSSSS | 1713 | SEQ ID NO: 160 |
| ChDC-F317 | 0.68 | KLELETTSNSKI | 2529 | SEQ ID NO: 161 |
| ChDC-F317 | 0.68 | LELETTSNSKIS | 2530 | SEQ ID NO: 162 |
| ChDC-F317 | 0.68 | ELETTSNSKISL | 2531 | SEQ ID NO: 163 |
| ChDC-F317 | 0.68 | LETTSNSKISLG | 2532 | SEQ ID NO: 164 |
| ChDC-F317 | 0.68 | IENKGGQITSES | 2557 | SEQ ID NO: 165 |
| ChDC-F317 | 0.68 | ENKGGQITSESG | 2558 | SEQ ID NO: 166 |
| ChDC-F317 | 0.68 | NKGGQITSESGA | 2559 | SEQ ID NO: 167 |
| ChDC-F317 | 0.68 | KGGQITSESGAT | 2560 | SEQ ID NO: 168 |
| ChDC-F317 | 0.68 | INLGNGSVGLYS | 2601 | SEQ ID NO: 169 |
| ChDC-F317 | 0.68 | NLGNGSVGLYSK | 2602 | SEQ ID NO: 170 |
| ChDC-F317 | 0.68 | LGNGSVGLYSKG | 2603 | SEQ ID NO: 171 |
| ChDC-F317 | 0.68 | GNGSVGLYSKGQ | 2604 | SEQ ID NO: 172 |
| ChDC-F317 | 0.68 | NGSVGLYSKGQS | 2605 | SEQ ID NO: 173 |
| ChDC-F317 | 0.68 | GSVGLYSKGQSN | 2606 | SEQ ID NO: 174 |
| ChDC-F317 | 0.68 | SVGLYSKGQSNT | 2607 | SEQ ID NO: 175 |
| Fn3-1-27 | 0.68 | NTSDKGNTNLDG | 934 | SEQ ID NO: 176 |
| Fn3-1-27 | 0.68 | TSDKGNTNLDGN | 935 | SEQ ID NO: 177 |
| Fn3-1-27 | 0.68 | NESNQANTQSEV | 1318 | SEQ ID NO: 178 |
| Fn3-1-27 | 0.68 | ESNQANTQSEVT | 1319 | SEQ ID NO: 179 |
| Fn3-1-27 | 0.68 | ESTAVGKGNVSA | 1872 | SEQ ID NO: 180 |
| Fn3-1-27 | 0.68 | STAVGKGNVSAE | 1873 | SEQ ID NO: 181 |
| Fn3-1-27 | 0.68 | IENKGGQITSES | 2566 | SEQ ID NO: 182 |
| Fn3-1-27 | 0.68 | ENKGGQITSESG | 2567 | SEQ ID NO: 183 |
| Fn3-1-27 | 0.68 | NKGGQITSESGA | 2568 | SEQ ID NO: 184 |
| Fn3-1-27 | 0.68 | KGGQITSESGAT | 2569 | SEQ ID NO: 185 |
| Fn3-1-36A2 | 0.68 | SVVNQETGISNL | 678 | SEQ ID NO: 186 |
| Fn3-1-36A2 | 0.68 | VVNQETGISNLP | 679 | SEQ ID NO: 187 |
| Fn3-1-36A2 | 0.68 | VNQETGISNLPN | 680 | SEQ ID NO: 188 |
| Fn3-1-36A2 | 0.68 | NQETGISNLPNA | 681 | SEQ ID NO: 189 |
| Fn3-1-36A2 | 0.68 | VNTSDKGNTNLD | 933 | SEQ ID NO: 190 |
| Fn3-1-36A2 | 0.68 | NTSDKGNTNLDG | 934 | SEQ ID NO: 191 |
| Fn3-1-36A2 | 0.68 | NESNQANTQSEV | 1318 | SEQ ID NO: 192 |
| Fn3-1-36A2 | 0.68 | ESNQANTQSEVT | 1319 | SEQ ID NO: 193 |
| Fn3-1-36A2 | 0.68 | SNQANTQSEVTN | 1320 | SEQ ID NO: 194 |
| Fn3-1-36A2 | 0.68 | IENKGGQITSES | 2566 | SEQ ID NO: 195 |
| Fn3-1-36A2 | 0.68 | ENKGGQITSESG | 2567 | SEQ ID NO: 196 |
| Fn3-1-36A2 | 0.68 | VGLYSKGQSYTI | 2617 | SEQ ID NO: 197 |
| Fn3-1-36A2 | 0.68 | GLYSKGQSYTIR | 2618 | SEQ ID NO: 198 |
| Fn3-1-36A2 | 0.68 | LYSKGQSYTIRN | 2619 | SEQ ID NO: 199 |
| Fn3-1-36A2 | 0.68 | KQINDKISSDPE | 3262 | SEQ ID NO: 200 |
| Fn3-1-36A2 | 0.68 | QINDKISSDPEG | 3263 | SEQ ID NO: 201 |
| Fn3-1-36A2 | 0.68 | INDKISSDPEGQ | 3264 | SEQ ID NO: 202 |
| Fn3-1-36A2 | 0.68 | NDKISSDPEGQA | 3265 | SEQ ID NO: 203 |
| Fn3-1-36A2 | 0.68 | DKISSDPEGQAL | 3266 | SEQ ID NO: 204 |
| Fn4-8 | 0.68 | KNASSQANTQSD | 1538 | SEQ ID NO: 205 |
| Fn4-8 | 0.68 | NASSQANTQSDV | 1539 | SEQ ID NO: 206 |
| Fn4-8 | 0.68 | ASSQANTQSDVT | 1540 | SEQ ID NO: 207 |
| Fn4-8 | 0.68 | SSQANTQSDVTN | 1541 | SEQ ID NO: 208 |
| Fn4-8 | 0.68 | SQANTQSDVINS | 1542 | SEQ ID NO: 209 |
| Fn4-8 | 0.68 | VENDNSITTKEE | 1756 | SEQ ID NO: 210 |
| Fn4-8 | 0.68 | ENDNSITTKEET | 1757 | SEQ ID NO: 211 |
| Fn4-8 | 0.68 | NDNSITTKEETS | 1758 | SEQ ID NO: 212 |
| Fn4-8 | 0.68 | DNSITTKEETSA | 1759 | SEQ ID NO: 213 |
| Fn4-8 | 0.68 | NSITTKEETSAG | 1760 | SEQ ID NO: 214 |
| Fn4-8 | 0.68 | SITTKEETSAGM | 1761 | SEQ ID NO: 215 |
| Fn4-8 | 0.68 | ITTKEETSAGMY | 1762 | SEQ ID NO: 216 |
| Fn4-8 | 0.68 | TTKEETSAGMYV | 1763 | SEQ ID NO: 217 |
| Fn4-8 | 0.68 | TKEETSAGMYVK | 1764 | SEQ ID NO: 218 |
| Fn4-8 | 0.68 | KEETSAGMYVKN | 1765 | SEQ ID NO: 219 |
| Fn4-8 | 0.68 | EETSAGMYVKNG | 1766 | SEQ ID NO: 220 |
| Fn4-8 | 0.68 | ETSAGMYVKNGN | 1767 | SEQ ID NO: 221 |
| Fn4-8 | 0.68 | ESTAVGKGNVSA | 1867 | SEQ ID NO: 222 |
| Fn4-8 | 0.68 | LKDSTVSNGSSA | 2132 | SEQ ID NO: 223 |
| Fn4-8 | 0.68 | KDSTVSNGSSAV | 2133 | SEQ ID NO: 224 |
| Fn4-8 | 0.68 | ENKGGQITSESG | 2563 | SEQ ID NO: 225 |
| Fn4-8 | 0.68 | NKGGQITSESGA | 2564 | SEQ ID NO: 226 |
| Fn4-8 | 0.68 | KGGQITSESGAT | 2565 | SEQ ID NO: 227 |
| Fn4-8 | 0.68 | SVGLYSKGQSYT | 2611 | SEQ ID NO: 228 |
| Fn4-8 | 0.68 | VGLYSKGQSYTV | 2612 | SEQ ID NO: 229 |
| Fn4-8 | 0.68 | GLYSKGQSYTVR | 2613 | SEQ ID NO: 230 |
| Fn4-8 | 0.68 | LYSKGQSYTVRN | 2614 | SEQ ID NO: 231 |
| Fn4-8 | 0.68 | YSKGQSYTVRNS | 2615 | SEQ ID NO: 232 |
| Fn4-8 | 0.68 | SKGQSYTVRNSV | 2616 | SEQ ID NO: 233 |
| Fn4-8 | 0.68 | KGQSYTVRNSVT | 2617 | SEQ ID NO: 234 |
| Fn4-8 | 0.68 | VNKTNIYNNTNT | 2975 | SEQ ID NO: 235 |
| Fn4-8 | 0.68 | NKTNIYNNTNTG | 2976 | SEQ ID NO: 236 |
| KCOM-1322 | 0.68 | KEEESVGMYSSS | 1712 | SEQ ID NO: 237 |
| KCOM-1322 | 0.68 | EEESVGMYSSSS | 1713 | SEQ ID NO: 238 |
| KCOM-1322 | 0.68 | KLELETTSNSKI | 2529 | SEQ ID NO: 239 |
| KCOM-1322 | 0.68 | LELETTSNSKIS | 2530 | SEQ ID NO: 240 |
| KCOM-1322 | 0.68 | ELETTSNSKISL | 2531 | SEQ ID NO: 241 |
| KCOM-1322 | 0.68 | LETTSNSKISLG | 2532 | SEQ ID NO: 242 |
| KCOM-1322 | 0.68 | IENKGGQITSES | 2557 | SEQ ID NO: 243 |
| KCOM-1322 | 0.68 | ENKGGQITSESG | 2558 | SEQ ID NO: 244 |
| KCOM-1322 | 0.68 | NKGGQITSESGA | 2559 | SEQ ID NO: 245 |
| KCOM-1322 | 0.68 | KGGQITSESGAT | 2560 | SEQ ID NO: 246 |
| KCOM-1322 | 0.68 | INLGNGSVGLYS | 2601 | SEQ ID NO: 247 |
| KCOM-1322 | 0.68 | NLGNGSVGLYSK | 2602 | SEQ ID NO: 248 |
| KCOM-1322 | 0.68 | LGNGSVGLYSKG | 2603 | SEQ ID NO: 249 |
| KCOM-1322 | 0.68 | GNGSVGLYSKGQ | 2604 | SEQ ID NO: 250 |
| KCOM-1322 | 0.68 | NGSVGLYSKGQS | 2605 | SEQ ID NO: 251 |
| KCOM-1322 | 0.68 | GSVGLYSKGQSN | 2606 | SEQ ID NO: 252 |
| KCOM-1322 | 0.68 | SVGLYSKGQSNT | 2607 | SEQ ID NO: 253 |
| KCOM-2931 | 0.68 | VVNQETGISNLP | 679 | SEQ ID NO: 254 |
| KCOM-2931 | 0.68 | VNQETGISNLPN | 680 | SEQ ID NO: 255 |
| KCOM-2931 | 0.68 | LNKGQLTENGVN | 771 | SEQ ID NO: 256 |
| KCOM-2931 | 0.68 | NKGQLTENGVNK | 772 | SEQ ID NO: 257 |
| KCOM-2931 | 0.68 | KGQLTENGVNKG | 773 | SEQ ID NO: 258 |
| KCOM-2931 | 0.68 | GQLTENGVNKGS | 774 | SEQ ID NO: 259 |
| KCOM-2931 | 0.68 | NESNQANTQSEV | 1318 | SEQ ID NO: 260 |
| KCOM-2931 | 0.68 | ESNQANTQSEVT | 1319 | SEQ ID NO: 261 |
| KCOM-2931 | 0.68 | SEVVNSERISLA | 1378 | SEQ ID NO: 262 |
| KCOM-2931 | 0.68 | EVVNSERISLAN | 1379 | SEQ ID NO: 263 |
| KCOM-2931 | 0.68 | VVNSERISLANN | 1380 | SEQ ID NO: 264 |
| KCOM-2931 | 0.68 | VNSERISLANNS | 1381 | SEQ ID NO: 265 |
| KCOM-2931 | 0.68 | NSERISLANNSI | 1382 | SEQ ID NO: 266 |
| KCOM-2931 | 0.68 | SERISLANNSIS | 1383 | SEQ ID NO: 267 |
| KCOM-2931 | 0.68 | ERISLANNSIST | 1384 | SEQ ID NO: 268 |
| KCOM-2931 | 0.68 | RISLANNSISTS | 1385 | SEQ ID NO: 269 |
| KCOM-2931 | 0.68 | ISLANNSISTSS | 1386 | SEQ ID NO: 270 |
| KCOM-2931 | 0.68 | SLANNSISTSSD | 1387 | SEQ ID NO: 271 |
| KCOM-2931 | 0.68 | IENKGGQITSES | 2566 | SEQ ID NO: 272 |
| KCOM-2931 | 0.68 | ENKGGQITSESG | 2567 | SEQ ID NO: 273 |
| KCOM-2931 | 0.68 | NKGGQITSESGA | 2568 | SEQ ID NO: 274 |
| KCOM-2931 | 0.68 | KGGQITSESGAT | 2569 | SEQ ID NO: 275 |
| MGYG-HGUT-01347 | 0.68 | SVVNQETGISNL | 678 | SEQ ID NO: 276 |
| MGYG-HGUT-01347 | 0.68 | VVNQETGISNLP | 679 | SEQ ID NO: 277 |
| MGYG-HGUT-01347 | 0.68 | VNQETGISNLPN | 680 | SEQ ID NO: 278 |
| MGYG-HGUT-01347 | 0.68 | NQETGISNLPNA | 681 | SEQ ID NO: 279 |
| MGYG-HGUT-01347 | 0.68 | VNTSDKGNTNLD | 933 | SEQ ID NO: 280 |
| MGYG-HGUT-01347 | 0.68 | NTSDKGNTNLDG | 934 | SEQ ID NO: 281 |
| MGYG-HGUT-01347 | 0.68 | NESNQANTQSEV | 1318 | SEQ ID NO: 282 |
| MGYG-HGUT-01347 | 0.68 | ESNQANTQSEVT | 1319 | SEQ ID NO: 283 |
| MGYG-HGUT-01347 | 0.68 | SNQANTQSEVTN | 1320 | SEQ ID NO: 284 |
| MGYG-HGUT-01347 | 0.68 | IENKGGQITSES | 2566 | SEQ ID NO: 285 |
| MGYG-HGUT-01347 | 0.68 | ENKGGQITSESG | 2567 | SEQ ID NO: 286 |
| MGYG-HGUT-01347 | 0.68 | VGLYSKGQSYTI | 2617 | SEQ ID NO: 287 |
| MGYG-HGUT-01347 | 0.68 | GLYSKGQSYTIR | 2618 | SEQ ID NO: 288 |
| MGYG-HGUT-01347 | 0.68 | LYSKGQSYTIRN | 2619 | SEQ ID NO: 289 |
| MGYG-HGUT-01347 | 0.68 | KQINDKISSDPE | 3262 | SEQ ID NO: 290 |
| MGYG-HGUT-01347 | 0.68 | QINDKISSDPEG | 3263 | SEQ ID NO: 291 |
| MGYG-HGUT-01347 | 0.68 | INDKISSDPEGQ | 3264 | SEQ ID NO: 292 |
| MGYG-HGUT-01347 | 0.68 | NDKISSDPEGQA | 3265 | SEQ ID NO: 293 |
| MGYG-HGUT-01347 | 0.68 | DKISSDPEGQAL | 3266 | SEQ ID NO: 294 |

To predict T cell epitopes, NetMHCpan 4.1 was used predict binding of all 8-11mer peptides derived from the nine F. nucleatum subsp. Fap2 reference sequences to all available human MHC (coded by HLA genes; n=2915). Peptide-MHC pairs with predicted IC50 values <500 nM were classified as binders. Each unique predicted binding peptide is shown in SEQ ID NOs: 317-4557, and a list of the F. nucleatum subsp. that contain that peptide in their Fap2 protein, and the list of HLA that are predicted to present that peptide is provided in U.S. provisional patent application No. 63/384,320 filed 18 Nov. 2022, the entirety of which is incorporated by reference herein.

### Example 9.0

### Preparation of Monoclonal Antibodies Directed Against Fap2

The inventors have obtained splenocytes from mice vaccinated with Fap2 antigens. Such splenocytes can be used as a source for the derivation of monoclonal antibodies directed against Fap2.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

### REFERENCES

The following references are of interest with respect to the subject matter described herein. Each of the following references is incorporated by reference in its entirety herein.
1. Bolstad, A., Jensen, H., and Bakken, V. 1996. Taxonomy, biology, and periodontal aspects of Fusobacterium nucleatum. Clin. Microbiol. Rev. 9:55-71.
2. Parhi, L., Abed, J., Shhadeh, A., Alon-Maimon, T., Udi, S., Ben-Arye, S.L., Tam, J., Parnas, O., Padler-Karavani, V., Goldman-Wohl, D., Yagel, S., Mandelboim, O., and Bachrach, G. 2022. Placental colonization by Fusobacterium nucleatum is mediated by binding of the Fap2 lectin to placentally displayed Gal-GalNAc. Cell Reports. 38 (12): 110537
3. Han, X., Weinberg, J., Prabhu, S., Hassenbusch, S., Fuller, G., and Tarrand, J. 2003. Fusobacterial brain abscess: a review of five cases and an analysis of possible pathogenesis. J. Neurosurg. 99:693-700.
4. Han, Y. W. 2015. Fusobacterium nucleatum: a commensal-turned pathogen. Curr. Opin. Microbiol. 23:141-147.
5. He Z, Tian W, Wei Q and Xu J. 2022. Involvement of Fusobacterium nucleatum in malignancies except for colorectal cancer: A literature review. Front. Immunol. 13:968649. doi: 10.3389/fimmu.2022.968649
6. Herrera L A et al. Role of infectious diseases in human carcinogenesis. Environmental & Molecular Mutagenesis. 2005; 45 (284-303)).
7. Kai, A., Cooke, F., Antoun, N., Siddharthan, C., and Sule, O. 2008. A rare presentation of ventriculitis and brain abscess caused by Fusobacterium nucleatum. J. Med. Microbiol. 57:668-671.
8. Krisanaprakornkit, S. and Dale, D. 2000. Inducible expression of human beta-defensin 2 by Fusobacterium nucleatum in oral epithelial cells: Multiple signaling pathways and role of commensal bacteria in innate immunity and the epithelial barrier. Infect. Immun. 68:2907-2915.
9. McLean M H, Murray G I, Stewart K N, Norrie G, Mayer C, Hold G L, Thomson J, Fyfe N, Hope M, Mowat N A et al. 2011. The Inflammatory Microenvironment in Colorectal Neoplasia. PLOS One 6: e15366.
10. Parkin, D. 2006. The global health burden of infection-associated cancers in the year 2002. Int. J. Cancer 118:3030-3044.
11. Peyret-Lacombe, A., Brunel, G., Watts, M., Charveron, M., and Duplan, H. 2009. TLR2 sensing of F. nucleatum and S. sanguinis distinctly triggered gingival innate response. Cytokine 46:201-210.
12. Serna, G. et al. 2020. Fusobacterium nucleatum persistence and risk of recurrence after preoperative treatment in locally advanced rectal cancer. Annals of Oncology 31 (10): 1366-1375.
13. Signat, B. and Duffaut, D. 2011. Role of Fusobacterium nucleatum in Periodontal Health and Disease. Curr. Issues Mol. Biol. 13:25-35.
14. Strauss, J., Kaplan, G. G., Beck, P. L., Rioux, K., Panaccione, R., DeVinney, R., Lynch, T., and Allen-Vercoe, E. 2011. Invasive potential of gut mucosa-derived Fusobacterium nucleatum positively correlates with IBD status of the host. Inflammatory Bowel Diseases. Advance online Publication.
15. Strauss, J., White, A., Ambrose, C., McDonald, J., and Allen-Vercoe, E. 2008. Phenotypic and genotypic analyses of clinical Fusobacterium nucleatum and Fusobacterium periodonticum isolates from the human gut. Anaerobe 14:301-309.
16. Swidsinski, A. and Ismail, M. 2011. Acute appendicitis is characterised by local invasion with Fusobacterium nucleatum/necrophorum. Gut 60:34-40.
17. Vogelstein, B. and Leppert, M. 19PCT88. Genetic alterations during colorectal-tumour development. N. Engl. J. Med. 319:525-532.
18. Weeks, D. F., Katz, D. S., Saxon, P. and Kubal, W. S. Lemierre syndrome: report of five new cases and literature review. 2010 Emerg. Radiol. 17:323-328.
19. Weiss, E. and Metzger, Z. 2000. Attachment of Fusobacterium nucleatum PK1594 to mammalian cells and its coaggregation with periodontopathogenic bacteria are mediated by the same galactose-binding adhesin. Oral Microbiol. Immunol. 15:371-377.
20. Wu S, Rhee K J, Albesiano E, Rabizadeh S, Wu X, Yen H R, Huso D L, Brancati FL, Wick E, McAllister F et al. 2009. A human colonic commensal promotes colon tumourigenesis via activation of T helper type 17 T cell responses. Nat Med 15:1016-1022.
21. Ximenez-Fyvie, L. and Socransky, S. 2000. Comparison of the microbiota of supra-and subgingival plaque in health and periodontitis. J. Clin. Periodontol. 27:648-657.
22. Yu, T. et al. 2017. Fusobacterium nucleatum promotes chemoresistance to colorectal cancer by modulating autophagy. Cell 170:548-563.

1. A target antigen comprising a Fap2 antigen, wherein the Fap2 antigen comprises a Fap2 passenger domain from Fusobacterium spp. or an antigenic fragment thereof.

2. The target antigen as defined in claim 1, comprising between 8 and 3500 contiguous amino acid residues of the extracellular passenger domain of Fap2.

3. The target antigen as defined in claim 1, comprising:
between 8 and 546 contiguous amino acid residues of the extracellular passenger domain of Fap2 extending between positions 1081 and 1627 of the reference Fap2 protein sequence from F. nucleatum 7/1; or
between 12 and 1256 contiguous amino acid residues of the extracellular passenger domain of Fap2 extending between positions 371 and 1627 of the reference Fap2 protein sequence from F. nucleatum 7/1.

4. The target antigen as defined in claim 1 having the amino acid sequence of any one of the FL, T1, T2, T3 or T4 constructs corresponding respectively to amino acid residues 22-3474, 22-350, 22-1059, 22-1606 or 22-2252 of SEQ ID NO: 1.

5. The target antigen as defined in claim 1, having the amino acid sequence of any one of FLΔT4, FLΔT3, FLΔT2 or FLΔT1, T4ΔT3, T4ΔT2, T4ΔT1, T3ΔT2, T3ΔT1 or T2ΔT1 corresponding respectively to amino acid residues 2253-3474, 1607-3474, 1060-3474, 351-3474, 1607-2252, 1060-2252, 351-2252, 1060-1606, 351-1606 or 351-1059 of SEQ ID NO:1.

6. The target antigen as defined in claim 1 that is a B-cell epitope, optionally wherein the B-cell epitope has the amino acid sequence of one of SEQ ID NOs: 127-294.

7. The target antigen as defined in claim 1 that is a T-cell epitope, optionally wherein the T-cell epitope has the amino acid sequence of one of SEQ ID NOs: 317-4557.

8. The target antigen as defined in claim 1, further comprising an N-terminal secretion signal.

9. The target antigen as defined in claim 8, wherein the N-terminal secretion signal comprises chymotrypsinogen, trypsinogen-2, interleukin-2, serum albumin preproprotein, immunoglobulin heavy chain, immunoglobulin light chain, azurocidin preproprotein, cystatin-S precursor, Ig kappa light chain precursor (mutant A2), oncostatin-M, glycoprotein G, Ig kappa chain V-III, Ig heavy chain V, SPARC, secrecon, Ig kappa chain V-I, myeloid cell surface antigen CD33, tissue-type plasminogen activator, gaussia luciferase, influenza haemagglutinin, insulin, or silkworm fibroin light chain.

10. The target antigen as defined in either claim 8, wherein the N-terminal secretion signal comprises one of SEQ ID NOs: 295-316.

11. The target antigen as defined in claim 8, wherein the N-terminal secretion signal comprises an Ig kappa signal peptide.

12. The target antigen as defined in claim 1, further comprising a transmembrane domain.

13. The target antigen as defined in claim 1, further comprising a C-terminal multimerization domain.

14. The target antigen as defined in claim 13, wherein the C-terminal multimerization domain comprises a self-assembling domain.

15. The target antigen as defined in claim 12, wherein the transmembrane domain or the self-assembling domain comprises IMX313, T3 (10), O3 (33), Nsp10, Lumazine Synthase, M1 VLP, I3 (01), I52 (32), I53 (50), I32 (28), HbsAg VLP, PDGFR, B7-1, CD28, CD8, CD86, FasL, IgM, Foldon, Ferritin, E2p, mi3, or AP205.

16. The target antigen as defined in claim 12, wherein the transmembrane domain comprises a transmembrane anchor derived from PDGFR or B7-1; or wherein the self-assembling domain comprises Foldon, Ferritin, E2p, mi3, AP205 or IMX313.

17. The target antigen as defined in claim 1 comprising the amino acid sequence of any one of SEQ ID NOs: 1-5, 46-80, 97-112, 120-126 or 4560-4562.

18. The target antigen as defined in claim 1 consisting of an isolated polypeptide having the amino acid sequence of any one of:
constructs FL, T1, T2, T3 or T4 corresponding respectively to amino acid residues 22-3474, 22-350, 22-1059, 22-1606 or 22-2252 of SEQ ID NO:1;
constructs FLΔT4, FLΔT3, FLΔT2 or FLΔT1, T4ΔT3, T4ΔT2, T4ΔT1, T3ΔT2, T3ΔT1 or T2ΔT1 corresponding respectively to amino acid residues 2253-3474, 1607-3474, 1060-3474, 351-3474, 1607-2252, 1060-2252, 351-2252, 1060-1606, 351-1606 or 351-1059 of SEQ ID NO: 1;
between 8 and 3500 contiguous amino acid residues of the extracellular passenger domain of Fap2;
between 8 and 546 contiguous amino acid residues the extracellular passenger domain of Fap2 extending between positions 1081 and 1627 of the reference Fap2 protein sequence from F. nucleatum 7/1; or
between 8 and 1256 contiguous amino acid residues of the extracellular passenger domain of Fap2 extending between positions 371 and 1627 of the reference Fap2 protein sequence from F. nucleatum 7/1;
SEQ ID NOs: 127-294; or
SEQ ID NOs: 317-4557.

19. The target antigen as defined in claim 1, wherein the target antigen has along its length an amino acid sequence having at least 90% sequence identity, and up to 100% sequence identity, to a corresponding portion of SEQ ID NO:1.

20. An isolated nucleic acid molecule comprising a sequence encoding the target antigen as defined in claim 1.

21. An isolated nucleic acid molecule consisting of a sequence encoding the target antigen as defined in claim 1.

22. The isolated nucleic acid molecule as defined in claim 20, wherein the nucleic acid comprises DNA or mRNA.

23. The isolated nucleic acid molecule as defined in claim 20 comprising or consisting of the nucleotide sequence of any one of SEQ ID NOs: 6-10, 11-45, 81-96, or 113-119.

24. A vaccine comprising the target antigen as defined in claim 1.

25. A vaccine comprising a nucleotide construct encoding the target antigen as defined in claim 1, wherein the nucleotide construct optionally comprises DNA or mRNA.

26. The vaccine as defined in claim 25, wherein the nucleotide construct is mRNA, and wherein the mRNA is formulated in a lipid nanoparticle.

27. The vaccine as defined in claim 24 comprising a viral vector vaccine or a DNA plasmid vaccine.

28. An antibody targeting the target antigen as defined in claim 1.

29. An antibody produced using the target antigen as defined in claim 1.

30. Use of the target antigen, the nucleic acid molecule, the vaccine or the antibody as defined in claim 1 to induce an immunological response against Fusobacterium spp. in a subject.

31. The use as defined in claim 30 to prevent or treat a cancer.

32. A method of inducing an immunological response against Fusobacterium spp. in a subject, the method comprising administering the target antigen, the nucleic acid molecule, the vaccine or the antibody as defined in claim 1 to the subject.

33. A method of preventing or treating a cancer in a subject, the method comprising administering the target antigen, the nucleic acid molecule, the vaccine or the antibody as defined in claim 1 to a subject.

34. The use or method as defined in claim 30 for preventing or mitigating chemoresistance of Fusobacterium ssp. positive cancers.

35. The use or method as defined in claim 30 for preventing re-colonization of a cancer with Fusobacterium ssp.

36. The use or method as defined in claim 30 for extending cancer remission in a patient that has received treatment for the cancer.

37. The use or method as defined in claim 30 for preventing metastatic spread of localized cancer by Fusobacterium ssp.

38. A method of treating cancer in a subject, the method comprising:
administering a cancer therapy to the patient; and
concurrently with or after administering the cancer therapy, administering the target antigen, the nucleic acid molecule, the vaccine or the antibody as defined in claim 1 to the subject.

39. The method as defined in claim 38, wherein the target antigen, the nucleic acid molecule, the vaccine or the antibody is provided as an adjuvant therapy or as a neo-adjuvant therapy for the cancer therapy.

40. The use or method as defined in claim 31, wherein the cancer is colorectal cancer (CRC), oral squamous cell carcinoma, oral/head or neck cancer, head and neck squamous cell carcinoma, esophageal cancer, esophageal squamous cell carcinoma, human papillomavirus positive oropharyngeal squamous cell carcinoma, gastric cardia adenocarcinoma, gastric cancer, Helicobacter pylori-positive gastric cancer, pancreatic cancer, stomach cancer, breast cancer, bladder cancer, cervical cancer, laryngeal squamous cell carcinoma, lung cancer, biliary tract cancer, or melanoma.

41. The use or method as defined in claim 31, wherein the cancer is a gastrointestinal cancer.

42. The use or method as defined in claim 41, wherein the gastrointestinal cancer is colorectal cancer.

43. The use or method as defined in claim 30 to prevent adverse pregnancy outcomes.

44. The use or method as defined in claim 43 wherein the adverse pregnancy outcomes are pre-term birth or miscarriages.

45. The use or method as defined in claim 43, wherein the adverse pregnancy outcomes are chorioamnionitis, neonatal sepsis, or preeclampsia.

46. The use or method as defined in claim 30 to prevent or treat a dental disease.

47. The use or method as defined in claim 46 wherein the dental disease is periodontitis.

48. The use or method as defined in claim 30 to prevent or treat an autoimmune disease.

49. The use or method as defined in claim 48 wherein the autoimmune disease is irritable bowel syndrome, atherosclerotic disease or rheumatoid arthritis.

50. The use or method as defined in claim 30 to prevent or treat a condition caused by or related to infection by Fusobacterium spp.

51. The use or method as defined in claim 50 wherein the condition caused by or related to direct infection by Fusobacterium spp. is appendicitis, sepsis or tissue abscesses.

52. The use or method as defined in claim 30 comprising, prior to or concurrently with administering the target antigen, the nucleic acid molecule, the vaccine or the antibody as defined in any one of the preceding claims, administering to the subject an antibiotic to treat or reduce an infection and/or colonization of Fusobacterium spp. in the subject, wherein the antibiotic optionally comprises metronidazole.

53. The use or method as defined in claim 30, wherein the immunological response comprises any one of inducing production of Fap2-specific neutralizing antibodies, or evoking a CD8+ T cell response to target Fusobacterium spp. invaded host cells.

54. A method of preventing immunosuppression by Fusobacterium spp. mediated by Fap2 blockade of T cell immunoreceptors with Ig and ITIM domains (TIGIT) in a subject, the method comprising administering to the subject the target antigen, the nucleic acid molecule, the vaccine or the antibody as defined in claim 1.

55. A method of disrupting an interaction between Fap2 of Fusobacterium spp. and a GalGal-NAc within a mammalian subject, the method comprising administering to the subject the target antigen, the nucleic acid molecule, the vaccine or the antibody as defined in claim 1.

56. The use or method as defined in claim 30, wherein the Fusobacterium spp. is F. nucleatum.

57. The use or method as defined in claim 30, wherein the Fusobacterium spp. is F. nucleatum 7/1, F. nucleatum ATCC23726, F. nucleatum ChDC-F317, F. nucleatum Fn3-1-27, F. nucleatum Fn3-1-36A2, F. nucleatum Fn4-8, F. nucleatum Fn71, F. nucleatum KCOM-1322, F. nucleatum KCOM-2931, or F. nucleatum MGYG-HGUT-01347.

58. The use or method as defined in claim 30, wherein the subject is a mammal, optionally wherein the subject is a human.
